SEQUENCE LISTING (5 sequences)

SEQ ID NO: 1 - DNA, 2124 bp, SV40
atggataaagttttaaacagagaggaatctttgcagctaatggaccttctaggtcttgaaaggagtgcctgggggaatattcctctgatgagaaaggcatatttaaaaaaatgcaagaagtttcatcctgataaaggaggagatgaagaaaaaatgaagaaaatgaatactctgtacaagaaaatggaagatggagtaaaatatgctcatcaacctgactttggaggcttctgggatgcaactgagattccaacctatggaactgatgaatgggagcagtggtggaatgcctttaatgaggaaaacctgttttgctcaaaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatccattttctgttattgaggaaagtttgccaggtgggttaaaggagcatgattttaatccagaagaagcagaggaaactaaacaagtgtcctggaagcttgtaacagagtatgcaatggaaacaaaatgtgatgatgtgttgttattgcttgggatgtacttggaatttcagtacagttttgaaatgtgtttaaaatgtattaaaaaagaacagcccagccactataagtaccatgaaaagcattatgcaaatgctgctatatttgctgacagcaaaaaccaaaaaaccatatgccaacaggctgttgatactgttttagctaaaaagcgggttgatagcctacaattaactagagaacaaatgttaacaaacagatttaatgatcttttggataggatggatataatgtttggttctacaggctctgctgacatagaagaatggatggctggagttgcttggctacactgtttgttgcccaaaatggaatcagtggtgtatgactttttaaaatgcatggtgtacaacattcctaaaaaaagatactggctgtttaaaggaccaattgatagtggtaaaactacattagcagctgctttgcttgaattatgtggggggaaagctttaaatgttaatttgcccttggacaggctgaactttgagctaggagtagctattgaccagtttttagtagtttttgaggatgtaaagggcactggaggggagtccagagatttgccttcaggtcagggaattaataacctggacaatttaagggattatttggatggcagtgttaaggtaaacttagaaaagaaacacctaaataaaagaactcaaatatttccccctggaatagtcaccatgaatgagtacagtgtgcctaaaacactgcaggccagatttgtaaaacaaatagattttaggcccaaagattatttaaagcattgcctggaacgcagtgagtttttgttagaaaagagaataattcaaagtggcattgctttgcttcttatgttaatttggtacagacctgtggctgagtttgctcaaagtattcagagcagaattgtggagtggaaagagagattggacaaagagtttagtttgtcagtgtatcaaaaaatgaagtttaatgtggctatgggaattggagttttagattggctaagaaacagtgatgatgatgatgaagacagccaggaaaatgctgataaaaatgaagatggtggggagaagaacatggaagactcagggcatgaaacaggcattgattcacagtcccaaggctcatttcaggcccctcagtcctcacagtctgttcatgatcataatcagccataccacatttgtagaggttttacttgctttaaaaaacctcccacacctccccctgaacctgaaaca

SEQ ID NO: 2 - DNA, 2799 bp, Homo sapiens
atgactgagctgaaggcaaagggtccccgggctccccacgtggcgggcggcccgccctcccccgaggtcggatccccactgctgtgtcgcccagccgcaggtccgttcccggggagccagacctcggacaccttgcctgaagtttcggccatacctatctccctggacgggctactcttccctcggccctgccagggacaggacccctccgacgaaaagacgcaggaccagcagtcgctgtcggacgtggagggcgcatattccagagctgaagctacaaggggtgctggaggcagcagttctagtcccccagaaaaggacagcggactgctggacagtgtcttggacactctgttggcgccctcaggtcccgggcagagccaacccagccctcccgcctgcgaggtcaccagctcttggtgcctgtttggccccgaacttcccgaagatccaccggctgcccccgccacccagcgggtgttgtccccgctcatgagccggtccgggtgcaaggttggagacagctccgggacggcagctgcccataaagtgctgccccggggcctgtcaccagcccggcagctgctgctcccggcctctgagagccctcactggtccggggccccagtgaagccgtctccgcaggccgctgcggtggaggttgaggaggaggatggctctgagtccgaggagtctgcgggtccgcttctgaagggcaaacctcgggctctgggtggcgcggcggctggaggaggagccgcggctgtcccgccgggggcggcagcaggaggcgtcgccctggtccccaaggaagattcccgcttctcagcgcccagggtcgccctggtggagcaggacgcgccgatggcgcccgggcgctccccgctggccaccacggtgatggatttcatccacgtgcctatcctgcctctcaatcacgccttattggcagcccgcactcggcagctgctggaagacgaaagttacgacggcggggccggggctgccagcgcctttgccccgccgcggagttcaccctgtgcctcgtccaccccggtcgctgtaggcgacttccccgactgcgcgtacccgcccgacgccgagcccaaggacgacgcgtaccctctctatagcgacttccagccgcccgctctaaagataaaggaggaggaggaaggcgcggaggcctccgcgcgctccccgcgttcctaccttgtggccggtgccaaccccgcagccttcccggatttcccgttggggccaccgcccccgctgccgccgcgagcgaccccatccagacccggggaagcggcggtgacggccgcacccgccagtgcctcagtctcgtctgcgtcctcctcggggtcgaccctggagtgcatcctgtacaaagcggagggcgcgccgccccagcagggcccgttcgcgccgccgccctgcaaggcgccgggcgcgagcggctgcctgctcccgcgggacggcctgccctccacctccgcctctgccgccgccgccggggcggcccccgcgctctaccctgcactcggcctcaacgggctcccgcagctcggctaccaggccgccgtgctcaaggagggcctgccgcaggtctacccgccctatctcaactacctgaggccggattcagaagccagccagagcccacaatacagcttcgagtcattacctcagaagatttgtttaatctgtggggatgaagcatcaggctgtcattatggtgtccttacctgtgggagctgtaaggtcttctttaagagggcaatggaagggcagcacaactacttatgtgctggaagaaatgactgcatcgttgataaaatccgcagaaaaaactgcccagcatgtcgccttagaaagtgctgtcaggctggcatggtccttggaggtcgaaaatttaaaaagttcaataaagtcagagttgtgagagcactggatgctgttgctctcccacagccagtgggcgttccaaatgaaagccaagccctaagccagagattcactttttcaccaggtcaagacatacagttgattccaccactgatcaacctgttaatgagcattgaaccagatgtgatctatgcaggacatgacaacacaaaacctgacacctccagttctttgctgacaagtcttaatcaactaggcgagaggcaacttctttcagtagtcaagtggtctaaatcattgccaggttttcgaaacttacatattgatgaccagataactctcattcagtattcttggatgagcttaatggtgtttggtctaggatggagatcctacaaacacgtcagtgggcagatgctgtattttgcacctgatctaatactaaatgaacagcggatgaaagaatcatcattctattcattatgccttaccatgtggcagatcccacaggagtttgtcaagcttcaagttagccaagaagagttcctctgtatgaaagtattgttacttcttaatacaattcctttggaagggctacgaagtcaaacccagtttgaggagatgaggtcaagctacattagagagctcatcaaggcaattggtttgaggcaaaaaggagttgtgtcgagctcacagcgtttctatcaacttacaaaacttcttgataacttgcatgatcttgtcaaacaacttcatctgtactgcttgaatacatttatccagtcccgggcactgagtgttgaatttccagaaatgatgtctgaagttattgctgcacaattacccaagatattggcagggatggtgaaaccccttctctttcataaaaag

SEQ ID NO: 3 - DNA, 924 bp, Artificial Sequence
  107/402-T-progesterone receptor fusion protein coding sequence
tctagactaggatcccgcgaccggtaccatggtccttggaggtcgaaaatttaaaaagttcaataaagtcagagttgtgagagcactggatgctgttgctctcccacagccagtgggcgttccaaatgaaagccaagccctaagccagagattcactttttcaccaggtcaagacatacagttgattccaccactgatcaacctgttaatgagcattgaaccagatgtgatctatgcaggacatgacaacacaaaacctgacacctccagttctttgctgacaagtcttaatcaactaggcgagaggcaacttctttcagtagtcaagtggtctaaatcattgccaggttttcgaaacttacatattgatgaccagataactctcattcagtattcttggatgagcttaatggtgtttggtctaggatggagatcctacaaacacgtcagtgggcagatgctgtattttgcacctgatctaatactaaatgaacagcggatgaaagaatcatcattctattcattatgccttaccatgtggcagatcccacaggagtttgtcaagcttcaagttagccaagaagagttcctctgtatgaaagtattgttacttcttaatacaattcctttggaagggctacgaagtcaaacccagtttgaggagatgaggtcaagctacattagagagctcatcaaggcaattggtttgaggcaaaaaggagttgtgtcgagctcacagcgtttctatcaacttacaaaacttcttgataacttgcatgatcttgtcaaacaacttcatctgtactgcttgaatacatttatccagtcccgggcactgagtgttgaatttccagaaatgatgtctgaagttattgctggaaaccggtcgaccagctttgcaaagatggataaagttttaaacag

SEQ ID NO: 4 - DNA, 876 bp, Artificial Sequence
  107/402-T-progesterone receptor fusion protein coding sequence
ctgtggcatggtccttggaggtcgaaaatttaaaaagttcaataaagtcagagttgtgagagcactggatgctgttgctctcccacagccagtgggcgtttcaaatgaaagccaagccctaagccagagattcactttttcaccaggtcaagacatacagttgattccaccactgatcaacctgttaatgagcattgaaccagatgtgatctatgcaggacatgacaacacaaaacctgacacctccagttctttgctgacaagtcttaatcaactaggcgagaggcaacttctttcagtagtcaagtggtctaaatcattgccaggttttcgaaacttacatattgatgaccagataactctcattcagtattcttggatgagcttaatggtgtttggtctaggatggagatcctacaaacacgtcagtgggcagatgctgtattttgcacctgatctaatactaaatgaacagcggatgaaagaatcatcattctattcattatgccttaccatgtggcagatcccacaggagtttgtcaagcttcaagttagccaagaagagttcctctgtatgaaagtattgttacttcttaatacaattcctttggaagggctacgaagtcaaacccagtttgaggagatgaggtcaagctacattagagagctcatcaaggcaattggtttgaggcaaaaaggagttgtgtcgagctcacagcgtttctatcaacttacaaaacttcttgataacttgcatgatcttgtcaaacaacttcatctgtactgcttgaatacatttatccagtcccgggcactgagtgttgaatttccagaaatgatgtctgaagttattgctgccccctatggaactgatga

SEQ ID NO: 5 - DNA, 2955 bp, Artificial Sequence
  107/402-T-progesterone receptor fusion protein coding sequence
atggataaagttttaaacagagaggaatctttgcagctaatggaccttctaggtcttgaaaggagtgcctgggggaatattcctctgatgagaaaggcatatttaaaaaaatgcaaagagtttcatcctgataaaggaggagatgaagaaaaaatgaagaaaatgaatactctgtacaagaaaatggaagatggagtaaaatatgctcatcaacctgactttggaggcttctgggatgcaactgagattccaacctatggaactgatgaatgggagcagtggtggaatgcctttaatgaggaaaacctgttttgctcaaaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatccattttctgttattgaggaaagtttgccaggtgggttaaaggagcatgattttaatccagaagaagcagaggaaactaaacaagtgtcctggaagcttgtaacagagtatgcaatggaaacaaaatgtgatgatgtgttgttattgcttgggatgtacttggaatttcagtacagttttgaaatgtgtttaaaatgtattaaaaaagaacagcccagccactataagtaccatgaaaagcattatgcaaatgctgctatatttgctgacagcaaaaaccaaaaaaccatatgccaacaggctgttgatactgttttagctaaaaagcgggttgatagcctacaattaactagagaacaaatgttaacaaacagatttaatgatcttttggataggatggatataatgtttggttctacaggctctgctgacatagaagaatggatggctggagttgcttggctacactgtttgttgcccaaaatggaatcagtggtgtatgactttttaaaatgcatggtgtacaacattcctaaaaaaagatactggctgtttaaaggaccaattgatagtggtaaaactacattagcagctgctttgcttgaattatgtggggggaaagctttaaatgttaatttgcccttggacaggctgaactttgagctaggagtagctattgaccagtttttagtagtttttgaggatgtaaagggcactggaggggagtccagagatttgccttcaggtcagggaattaataacctggacaatttaagggattatttggatggcagtgttaaggtaaacttagaaaagaaacacctaaataaaagaactcaaatatttccccctggaatagtcaccatgaatgagtacagtgtgcctaaaacactgcaggccagatttgtaaaacaaatagattttaggcccaaagattatttaaagcattgcctggaacgcagtgagtttttgttagaaaagagaataattcaaagtggcattgctttgcttcttatgttaatttggtacagacctgtggctgagtttgctcaaagtattcagagcagaattgtggagtggaaagagagattggacaaagagtttagtttgtcagtgtatcaaaaaatgaagtttaatgtggctatgggaattggagttttagattggctaagaaacagtgatgatgatgatgaagacagccaggaaaatgctgataaaaatgaagatggtggggagaagaacatggaagactcagggcatgaaacaggcattgattcacagtcccaaggctcatttcaggccaaaaagttcaataaagtcagagttgtgagagcactggatgctgttgctctcccacagccagtgggcgttccaaatgaaagccaagccctaagccagagattcactttttcaccaggtcaagacatacagttgattccaccactgatcaacctgttaatgagcattgaaccagatgtgatctatgcaggacatgacaacacaaaacctgacacctccagttctttgctgacaagtcttaatcaactaggcgagaggcaacttctttcagtagtcaagtggtctaaatcattgccaggttttcgaaacttacatattgatgaccagataactctcattcagtattcttggatgagcttaatggtgtttggtctaggatggagatcctacaaacacgtcagtgggcagatgctgtattttgcacctgatctaatactaaatgaacagcggatgaaagaatcatcattctattcattatgccttaccatgtggcagatcccacaggagtttgtcaagcttcaagttagccaagaagagttcctctgtatgaaagtattgttacttcttaatacaattcctttggaagggctacgaagtcaaacccagtttgaggagatgaggtcaagctacattagagagctcatcaaggcaattggtttgaggcaaaaaggagttgtgtcgagctcacagcgtttctatcaacttacaaaacttcttgataacttgcatgatcttgtcaaacaacttcatctgtactgcttgaatacatttatccagtcccgggcactgagtgttgaatttccagaaatgatgtctgaagttattgctgcccctcagtcctcacagtctgttcatgatcataatcagccataccacatttgtagaggttttacttgctttaaaaaacctcccacacctccccctgaacctgaaacataa

(19) United States
(12) Patent Application Publication
(10) Pub. No.: US 2002/0031803 A1
(43) Pub. Date: Mar. 14, 2002

(54) Expression system for production of therapeutic proteins

(76) Inventor: Mark J. Cooper, Moreland Hills, OH (US)

Correspondence Address: BANNER & WITCOFF, 1001 G STREET N W, SUITE 1100, WASHINGTON, DC, 20001, US

(21) Appl. No.: 09/935,368

(22) Filed: Aug. 24, 2001

Related U.S. Application Data
(63) Continuation-in-part of application No. 09/473,646, filed on Dec. 28, 1999.
(60) Provisional application No. 60/050,356, filed on Jun. 20, 1997.

(30) Foreign Application Priority Data
Jun. 19, 1998 (US) PCT/US98/12777

(51) Int. Cl. C12P 21/02; C07K 14/705; C07H 21/04

(52) U.S. Cl. 435/69.1; 435/325; 435/320.1; 536/23.5; 530/350

(57) ABSTRACT

Novel methods are described for expression of biologically functional therapeutic and other proteins without host cell toxicity. The methods take advantage of the surprising replication activating ability of the 107/402-T antigen. The invention also provides fusion proteins, expression vectors, and mammalian cells, for practicing the methods.

This application is a continuation-in-part of copending application Ser. No. 09/473,646 filed Dec. 28, 1999, which claims the benefit of PCT/US98/12777 filed Jun. 19, 1998, which claims the benefit of Ser. No. 60/050,356, filed Jun. 20, 1997. Each application is incorporated herein by reference.

The U.S. Government has a paid-up license in this invention and the right in limited circumstances to require the patent owner to license others on reasonable terms as provided for by the terms of IR55CA/OD66780, CA72737, and IR43CA73376 awarded by the National Institutes of Health.

### TECHNICAL AREA OF THE INVENTION

The invention relates to the area of protein expression. More particularly, the invention relates to human systems for expressing proteins of therapeutic value.

### BACKGROUND OF THE INVENTION

The production of large quantities of biologically functional therapeutic proteins requires an expression system that can both produce protein efficiently without toxic effects to the expression system itself and perform the required post-translational modifications. One approach to in vitro protein production is to transfect a bacterial or yeast cell with a plasmid encoding the protein of interest and culture the cell under conditions where the plasmid replicates to a high copy number, resulting in the potential for the production of large amounts of the desired protein. Due to differences in the biology of bacterial, yeast, and human cells, however, many non-human expression systems have very low efficiencies of producing functional product when the desired protein requires post-translational modification to be functional (Yarranton, 1990; Geisse et al. 1996). In mammalian cells, where post-translational modification of the desired protein may be accomplished more effectively, plasmids encoding the protein of interest are often replicated under control of a replication activator such as the SV40 large T antigen. Although the SV40 large T antigen is an efficient replication activator, high levels of extrachromosomal DNA replicating under the control of SV40 large T antigen normally are toxic to host cells (Gerard and Gluzman, 1985). This toxicity results in expression systems which function for only a short time.

Thus there is a need in the art for new systems for producing functional proteins for therapeutic uses.

### SUMMARY OF THE INVENTION

It is an object of the invention to provide tools and methods for producing functional proteins for therapeutic uses. These and other objects of the invention are provided by one or more of the embodiments described below.

One embodiment of the invention is a fusion protein comprising a 107/402-T antigen and a truncated hormone binding domain of a progesterone receptor. The truncated hormone binding domain binds an antiprogestin but does not bind progesterone. The truncated hormone binding domain is covalently bound at its amino terminus to amino acid 673 of the 107/402-T antigen.

Another embodiment of the invention is an isolated polynucleotide comprising a first coding sequence for a 107/402-T antigen and a second coding sequence for a truncated hormone binding domain of a progesterone receptor. The truncated portion of the hormone binding domain binds an antiprogestin but does not bind progesterone. The 5′-most codon of the second coding sequence is located directly 3′ of codon 673 of the first coding sequence.

Even another embodiment of the invention is a transcription cassette comprising a first coding sequence for a 107/402-T antigen, a second coding sequence comprising codons 640-914 of a human progesterone receptor coding sequence, a promoter that controls transcription of the first and second coding sequences, an internal ribosome entry site, and a third coding sequence for a desired protein. The 5′-most codon of the second coding sequence is located directly 3′ of codon 673 of the first coding sequence.

Still another embodiment of the invention is an expression vector comprising a polynucleotide encoding a fusion protein. The polynucleotide comprises a first coding sequence for a 107/402-T antigen and a second coding sequence for a truncated hormone binding domain of a progesterone receptor. The truncated hormone binding domain binds an antiprogestin but does not bind progesterone, and the 5′-most codon of the second coding sequence is located directly 3′ of codon 673 of the first coding sequence.

A further embodiment of the invention is an expression vector comprising a polynucleotide encoding a fusion protein. The polynucleotide comprises a first coding sequence for a 107/402-T antigen, a second coding sequence comprising codons 640-914 of a human progesterone receptor coding sequence, wherein the 5′-most codon of the second coding sequence is located directly 3′ of codon 673 of the first coding sequence, a promoter that controls transcription of the first and second coding sequences, wherein the promoter is selected from the group consisting of an oncofetal promoter and a tissue-specific promoter, an internal ribosome entry site, a third coding sequence for a desired protein, and an SV40 origin of replication. Yet another embodiment of the invention is an isolated mammalian cell comprising this expression vector.

Another embodiment of the invention is an isolated mammalian cell comprising an expression vector. The expression vector comprises a polynucleotide encoding a fusion protein, wherein the polynucleotide comprises a first coding sequence for a 107/402-T antigen and a second coding sequence for a truncated hormone binding domain of a progesterone receptor, wherein the truncated hormone binding domain binds an antiprogestin but does not bind progesterone, and wherein the 5′-most codon of the second coding sequence is located directly 3′ of codon 673 of the first coding sequence.

Even another embodiment of the invention is a kit for expressing a desired protein, comprising an expression vector comprising a polynucleotide encoding a fusion protein. The polynucleotide comprises a first coding sequence for a 107/402-T antigen and a second coding sequence for a truncated hormone binding domain of a progesterone receptor. The truncated hormone binding domain binds an antiprogestin but does not bind progesterone, and the 5′-most codon of the second coding sequence is located directly 3′ of codon 673 of the first coding sequence.

Still another embodiment of the invention is a kit for expressing a desired protein, comprising a human cell comprising an expression vector, wherein the expression vector comprises a polynucleotide encoding a fusion protein. The polynucleotide comprises a first coding sequence for a 107/402-T antigen and a second coding sequence comprising codons 640-914 of a human progesterone receptor coding sequence. The 5′-most codon of the second coding sequence is located directly 3′ of codon 673 of the first coding sequence. The polynucleotide also comprises a promoter that controls transcription of the first and second coding sequences, wherein the promoter is selected from the group consisting of an oncofetal promoter and a tissue-specific promoter, an internal ribosome entry site, and a third coding sequence for a desired protein.

Yet another embodiment of the invention is a kit for expressing a desired protein, comprising a mammalian cell comprising an expression vector, wherein the expression vector comprises a polynucleotide encoding a fusion protein. The polynucleotide comprises a first coding sequence for a 107/402-T antigen and a second coding sequence for a truncated hormone binding domain of a progesterone receptor. The truncated hormone binding domain binds an antiprogestin but does not bind progesterone, and the 5′-most coding of the second coding sequence is located directly 3′ of codon 673 of the first coding sequence.

Yet another embodiment of the invention is a method of expressing a desired protein. A mammalian cell is cultured under conditions whereby the desired protein can be expressed. The mammalian cell comprises an expression vector comprising a polynucleotide comprising (1) a first coding sequence for a 107/402-T antigen, (2) a second coding sequence for a truncated hormone binding domain of a progesterone receptor, wherein the truncated hormone binding domain binds an antiprogestin but does not bind progesterone, wherein the 5′-most codon of the second coding sequence is located directly 3′ of codon 673 of the first coding sequence, and (3) a third coding sequence for the desired protein. The mammalian cell is contacted with an antiprogestin. The desired protein is thereby expressed.

Another embodiment of the invention is a method of expressing a desired protein. A mammalian cell is contacted with an antiprogestin. The mammalian cell comprises an expression vector comprising a polynucleotide comprising (1) a first coding sequence for a 107/402-T antigen, (2) a second coding sequence for a truncated hormone binding domain of a progesterone receptor, wherein the truncated hormone binding domain binds an antiprogestin but does not bind progesterone, wherein the 5′-most codon of the second coding sequence is located directly 3′ of codon 673 of the first coding sequence, and (3) a third coding sequence for the desired protein. The desired protein is thereby expressed.

Thus, the present invention provides the art with expression vectors, mammalian cells, and fusion proteins for practicing a method of producing therapeutic and other useful proteins in vitro and in vivo.

### BRIEF DESCRIPTION OF THE DRAWINGS

FIG. 1. FIG. 1 shows point mutations in replication-competent, safety modified SV40 large T antigen mutants. Domains of T antigen that bind to RB, p53, and the SV40 DNA origin are highlighted. The codon 107 mutation substitutes lysine for glutamic acid, and the codon 402 mutation substitutes glutamic acid for aspartic acid.

FIG. 2. FIG. 2 demonstrates the presence of point mutations in codons 107 and 402 of replication-competent safety-modified SV40 large T antigen mutants.

FIG. 3. FIG. 3 shows co-immunoprecipitation analysis of binding of wild-type and mutant T antigens to human tumor suppressor gene products. In vitro translated T antigen (2×10⁵ dpm) was mixed with CV-1 extracts over producing human RB protein and anti-RB monoclonal antibody G3-245 (FIG. 3A, lanes 3-6), p53, and anti-p53 monoclonal antibody 1801 (FIG. 3A lanes 7-10), and p107 and anti-p107 monoclonal antibody SD9 (FIG. 3B, lanes 3-6). As controls, wild-type T antigen is immunoprecipitated with either anti-chromogranin A monoclonal antibody LKH210 (lane 1 of FIG. 3A and FIG. 3B) or anti-T antigen monoclonal antibody 416 (lane 2 of FIG. 3A and FIG. 3B).

FIG. 4. FIG. 4 demonstrates that 107/402-T is replication-competent.

FIG. 4A. HepG2 hepatoma cells were transfected with wild-type and mutant T antigen expression vectors, and total DNA was harvested 2 days post-transfection. DNA samples were sequentially digested with ApaI to linearize vector DNA and then with DpnI to distinguish amplified DNA from the input DNA used to transfect these cells. Since human cells lack adenine methylase activity, newly replicated DNA is resistant to DpnI digestion. Hence, the presence of unit length, linearized plasmid DNA (as indicated by the arrow) demonstrates newly replicated episome. Hybridization probe: pRC/CMV.107/402-T.

FIG. 4B. Replication activity of wild-type SV40 large T antigen and SV40 T antigen mutants.

FIG. 5. FIG. 5 shows enhanced replication activity of 107/402-T in HepG2 cells.

FIGS. 5A and 5B show FACS analysis of propidium iodide-stained HepG2 cells.

FIG. 5C is a Southern blot which shows episomal copy number of wild-type and 107/402-T expression vectors in transfected HepG2 cells.

FIG. 5D shows normalized replication activity of 107/402-T and wild-type T antigen expression vectors.

FIG. 6. FIG. 6 illustrates transgene (alkaline phosphatase) expression mediated by 107/402-T or wild-type T antigen in transiently transfected HepG2 cells.

FIG. 7. FIG. 7 depicts the time course of induction of 107/402-T expression vectors in an HT-1376 tet-off clone by removal of doxycycline.

FIG. 8. FIG. 8 shows dependence of 107/402-T expression on doxycycline concentration. Cells were harvested for Western blot analysis of T antigen expression 4 days after exposure to doxycycline.

FIG. 9. FIG. 9 shows the half-life of 107/402-T expression after addition of 3 ng/ml of doxycycline.

FIG. 10.

FIG. 10A shows cyclic production of secreted alkaline phosphatase (SEAP).

FIG. 10B is a Western blot of protein extracts demonstrating 107/402-T antigen expression.

FIG. 11. Activation of 107/402-T/PR fusion proteins by RU486.

FIG. 12. Replication activity of 107/402-T/PR fusion protein in the presence of endogenous steroid hormones.

FIG. 13. Design of an externally-controlled replication switch vector.

FIG. 14. Southern blots of HepG2 cells transfected with PRdmT plasmid constructs in the presence and absence of RU486 stimulation (+/−).

FIG. 14A, Southern blot showing the replication of plasmid constructs with the progesterone receptor (PR) fragment inserted at amino acids 1 or 85 of the 107/402 double mutant SV40 T antigen (dmT).

FIG. 14B, Southern blot showing the replication of a plasmid construct with the progesterone receptor fragment inserted at amino acid 673 of the dmT.

FIG. 15. Sequencing of 107/402-T/PR fusion constructs.

FIG. 15A, sequence of pRcCMV1/PR632-914/dmT (insertion at amino acid 1; SEQ ID NO:3).

FIG. 15B, sequence of pRcCMV85/PR631-915/dmT (insertion at amino acid 85; SEQ ID NO:4).

FIG. 15C, sequence of pKCPIR673/PR640-914/dmT+ (insertion at amino acid 673; SEQ ID NO:5).

FIG. 16. In vitro translation of PRdmT constructs.

### DETAILED DESCRIPTION OF THE PREFERRED EMBODIMENTS

It is a discovery of the present invention that the mutant large T antigen, 107/402-T antigen, is an exceptionally efficient replication transactivator in human cells. This property of 107/402-T antigen can be employed in expression systems to produce proteins of therapeutic utility. Use of the 107/402-T antigen permits expression which continues for long periods of time and which produces large quantities of biologically active proteins.

The present invention overcomes significant limitations of the prior art. According to the present invention, human cells are genetically modified to produce very high levels of biologically functional proteins and to continue this production over long periods of time without significant cell toxicity. These human cells comprise copies of 107/402-T antigen which retain high levels of replication transactivator activity in dividing human cells. Preferably, the copies of the 107/402-T antigen are integrated. Surprisingly, 107/402-T antigen is an exceptionally efficient replication transactivator in human cells when compared with wild-type T antigen.

Also according to the present invention, either the production or activity of 107/402-T antigen in mammalian, including human, cells can be cyclically controlled by the presence of varying concentrations of exogenous agents in the culture medium. The method of cyclically controlling replication described herein permits amplification of an episome (i.e., an extrachromosomal element, such as an expression vector) to a level which yields high gene expression without induction of cellular toxicity. A desired protein can then be produced at high levels. Furthermore, because human cells can be used in this expression system, post-translational modification of the desired protein(s) proceeds normally. Thus, the present invention provides the art with an expression system for therapeutic proteins which is useful in the pharmaceutical and biotechnology industries.

The 107/402-T antigen mutant is described in U.S. Pat. No. 5,624,820. Compared with the wild-type SV40 large T antigen (see Shin et al., 1975; Christian et al., 1987; Michalovitz et al., 1987; DeCaprio et al., 1988; Hanahan et al., 1989; Chen et al., 1990; Chen et al., 1992), the mutant protein contains substitutions of amino acid residues 107 (glutamic acid to lysine) and 402 (aspartic acid to glutamic acid). These amino acid substitutions prevent the 107/402-T antigen from binding to the oncogenes p53, RB, and p107, yet the mutant antigen retains the ability to activate replication of a papovavirus-based episome. A nucleotide coding sequence for the 107/402-T antigen is shown in SEQ ID NO:1.

The 107/402-T antigen binds to the papovavirus origin of replication and activates the replication of adjacent DNA sequences. Under control of the 107/402-T antigen, papovavirus-based episomes replicate to thousands of copies by 2-4 days after transfection in many human cell lines. This replication is greatly enhanced compared with that observed in the presence of wild-type T antigen (Examples 3, 4, and 5). Under control of the 107/402-T antigen, episomal copy number can range from at least 2-, 5-, 10-, 25-, 50-, 100-, 125-, 150-, 200- or 500-fold higher than episomal copy number obtained under control of a wild-type T antigen.

Regulating Transcription of 107/402-T Antigen Using an Inducible Transcriptional Transregulator

In one embodiment of the present invention, replication of an episome encoding the protein to be expressed is controlled by regulating transcription of the 107/402-T DNA sequence. Transcription of the DNA sequence is controlled by a minimally active promoter, which can be activated by an inducible transcriptional transregulator. The minimally active promoter prevents large amounts of 107/402-T antigen from being transcribed in the absence of an exogenous inducer of the transcriptional transregulator. Suitable minimally active promoters are, for example, the minimal CMV promoter (Boshart et al., 1985) and the promoters for TK (Nordeen, 1988), IL-2, and MMTV.

An inducible transcriptional transregulator can be either a transactivator or a transrepressor. Several inducible transcriptional transactivators have been constructed, such as the hybrid tetracycline-controlled transcriptional transactivator (Gossen et al., 1992; Gossen et al. 1995), the rapamycin-controlled “gene switch” (Rivera et al., 1996), and the RU486-induced TAXI/UAS “molecular switch” (DeLort and Capecchi, 1996). Each transactivator contains a binding site for its inducer and a transcription factor domain. These inducible transcriptional transactivators bind reversibly to specific-binding regions of DNA, such as operators, and regulate an adjacent minimal promoter which is functional only when the transcription factor binds to the specific region of DNA.

Inducible repressor systems have also been developed by substituting the KRAB transcriptional repressor domain for the VP16 transactivation domain in hybrid transcription factors (Wang et al. 1997). In these systems, repression of gene transcription is linked to binding of the transcriptional repressor to the target DNA binding consensus sequence, and binding of the transcriptional repressor is controlled by suitable inducer molecules.

A transcriptional transregulator can be constructed to be either functional (“inducer-on”) or nonfunctional (“inducer-off”) in the presence of inducer. An “inducer-on” transcriptional transregulator is not functional in the absence of inducer. In the presence of inducer, the transcription factor domain of the “inducer-on” transcriptional transregulator binds to the specific-binding DNA region and activates the minimally active promoter. An “inducer-off” transcriptional transregulator functions in the absence of inducer. In the presence of inducer, the transcription factor domain of the “inducer-off” transcriptional transregulator does not bind to the specific-binding DNA region and does not activate the minimally active promoter. DNA sequences encoding either type of inducible transcriptional transregulator can be used to practice this invention.

DNA sequences encoding the 107/402-T antigen, a minimally active promoter, and an inducible transcriptional transregulator can be located on the same DNA construct or can be encoded by separate DNA constructs. Optionally, the DNA sequences encoding the transcriptional transregulator and the DNA sequence encoding the 107/402-T antigen can be on an episome. The episome can comprise a papovavirus origin of replication and a restriction enzyme site for insertion of a coding sequence of a desired protein. Alternatively, the papovavirus origin of replication and restriction enzyme site can be on an episome separate from the DNA constructs encoding the 107/402 antigen, the minimally active promoter, and the inducible transcriptional transregulator. The episome can also comprise a promoter which regulates transcription of the coding sequence of the desired protein. Individual DNA constructs or episomes can be introduced into a cell together or separately, as is desired.

Expression vectors can be constructed containing one or more copies of a particular DNA construct. Many suitable vectors are available from commercial suppliers, such as Stratagene, GIBCO-BRL, Amersham, and Promega, as well as from noncommercial sources such as the American Type Culture Collection (ATCC), 10801 University Blvd., Manassas, Va. 20110-2209. Suitable vectors may also be constructed in the laboratory using standard recombinant DNA techniques (Sambrook et al., 1989; Glover, 1985; Perbal, 1984). The sequences can be synthesized chemically or can produced by recombinant DNA methods.

Methods of transfecting DNA into human cells are well known in the art. These methods include, but are not limited to, transferrin-polycation-mediated DNA transfer, transfer with naked or encapsulated nucleic acids, liposome-mediated cell fusion, intracellular uptake of DNA-coated latex beads, protoplast fusion, viral infection, electroporation, and calcium phosphate-mediated transfection. Integration of the DNA sequences encoding the inducible transcription transregulator and the 107/402-T antigen into the host cell's DNA can be facilitated by providing nucleotides at the 3′ or 5′ ends of these DNA sequences which are homologous to and therefore recombine with the host cell DNA. One or more copies of each DNA sequence or episome can be integrated into the genome of the host cell, as desired.

The host cell can be any human cell. Preferably, the host cell is capable of dividing and being maintained in vitro, such as HT-1376 (bladder carcinoma), HepG2 (hepatoma), HEK 293 (human embryonic kidney), HT1080 (fibrosarcoma), HeLa (cervical carcinoma), Hs68 (fibroblasts), RAJI (lymphoma), SW480 (colon cancer), 5637 (bladder carcinoma), MCF-7 (breast carcinoma), or HuNS1 (myeloma) cells. Preferred host cells are those which are particularly well-suited for protein secretion, such as myeloma cell lines. Many of these cell lines, together with instructions on how to culture them, are available from the ATCC. Suitable methods for maintaining cell lines in culture are also well known in the art (see Freshney, 1986).

In addition to containing the minimally active promoter, the DNA sequences encoding the 107/402-T antigen, and an inducible transcriptional transregulator, the host cell can contain an episome. The episome comprises a papovavirus origin of replication, a DNA sequence encoding the desired protein to be expressed, a promoter which is functional in the host cell, and a multiple cloning site for insertion of the protein coding sequence, or transgene (see, for example, Walter and Blobel, 1982; Caras and Weddell, 1989). According to the invention, transgene expression can be increased at least 2-, 3-, 4-, or 5-fold or more over expression levels achieved using an expression vector encoding wild-type T antigen.

The protein encoded by the transgene or protein coding sequence can be, for example, any protein of therapeutic utility, including but not limited to a structural protein, an anti-angiogenic or pro-angiogenic factor, a transcription factor, a cytokine, a neuropeptide, a ligand for a cell surface receptor, an enzyme, a growth factor, a receptor for a ligand, an antibody, a hormone, a transport protein, a storage protein, a contractile protein, or a novel engineered protein. The protein can be one which is normally encoded by an endogenous gene in the host cell or can be a protein not normally found in the host cell. The protein can be identical to a naturally occurring protein or can contain modifications to alter its physicochemical properties, such as stability, activity, affinity for a particular ligand or receptor, antigenicity, therapeutic utility, or ability to be secreted from the host cell. The protein can also be a fusion protein comprising two or more protein fragments fused together by means of a peptide bond. The fusion protein can include signal peptide sequences to cause secretion of the protein into the culture medium. Such sequences are well known in the art.

The promoter can be any promoter which is functional in the selected host cell. Highly active promoters, such as the regulatory region of elongation factor-1α (Guo et al., 1996), are preferred. Multiple cloning sites are well known in the art and can be inserted into the episome using standard recombinant DNA techniques.

The episome also comprises a papovavirus origin of replication to which the 107/402-T antigen binds. In a preferred embodiment, the origin of replication is an SV40 or a BK origin of replication. The sequence of the SV40 origin of replication is taught in Subramanian et al., 1977; Reddy et al. 1978; Fiers et al., 1978; and Van Heuverswyn et al., 1978. The sequence of the BK origin of replication is disclosed in Yang et al. (1979) and Deyerle et al. (1989).

Those of skill in the art can select suitable episomes for use in this protein expression system from those available commercially or noncommercially, such as from the ATCC. Alternatively, one can synthesize an episome in the laboratory using standard recombinant DNA techniques. Episomes can also contain a selectable marker, such as the neomycin phosphotransferase gene or antibiotic resistance genes.

In one embodiment of the invention, the host cell is cultured in a medium which is suitable to maintain the particular cell type being used. The cell is contacted with an inducer of the inducible transcriptional transregulator. The inducer can be a component of the cell culture medium or can be added separately. In a preferred embodiment, the inducible transcriptional transregulator is a hybrid tetracycline-controlled transcriptional transactivator. Tetracycline or a tetracycline derivative such as oxytetracycline, chlortetracycline, anhydrotetracycline, or doxycycline, is added to the culture medium to cause the transactivator to regulate transcription of the DNA sequence encoding the 107/402-T antigen.

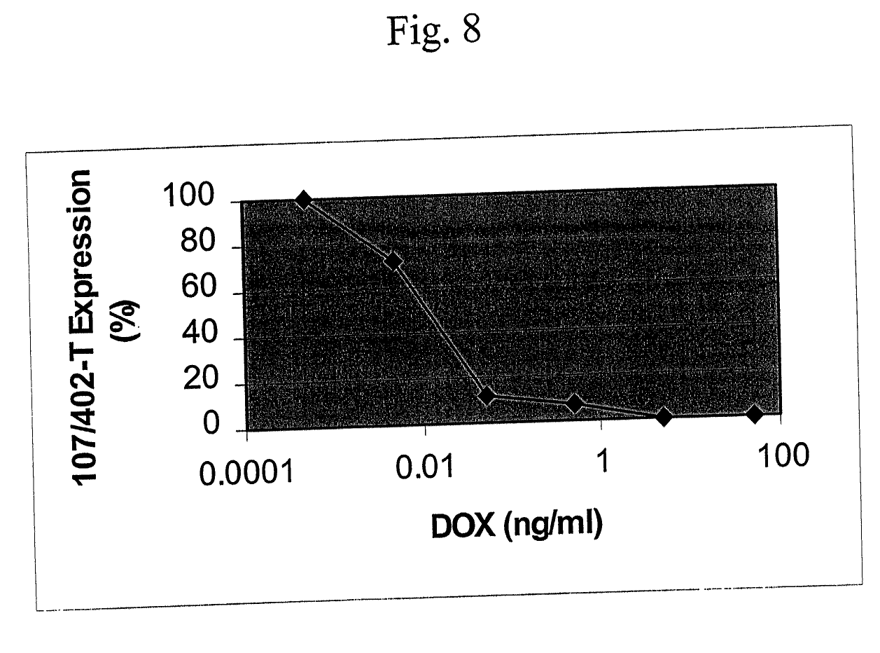

The concentration of inducer is selected by routine experimentation to result in an episome copy number for the particular cell line which results in maximal expression of the protein without cellular toxicity. Appropriate copy numbers range from at least 10 to at least 100, at least 100 to at least 1,000, at least 1,000 to at least 10,000, at least 10,000 to at least 50,000, at least 50,000 to at least 100,000, or at least 100,000 to at least 500,000 copies or more of the plasmid per cell. Plasmid copy number can be measured, for example, by Southern blot (Cooper and Miron, 1993). For tetracycline or its derivatives, effective concentrations range from at least 1 pg/ml to at least 1 μg/ml. For rapamycin, suitable concentrations range from at least 500 pM to at least 2 nM to at least 10 nM to at least 100 nM. The half-maximal concentration for inhibition using doxycycline, for example is approximately 0.01 ng/ml (FIG. 8). Concentrations of RU486 which can be used effectively range from at least 1 nM to at least 100 nM.

Inducer concentration can be varied over time to achieve suitable copy numbers per cell. For example, inducer can be present continuously for 1-3 days or for 1-6 days and then removed entirely, for example by changing the medium. Alternatively, medium can be changed every 2-3 days and the concentration of inducer can be varied, for example, by one-half or one-tenth. The precise variation regimen will depend on the cell being used and the stability of the inducer under particular culture conditions. These parameters can be determined by routine experimentation. Thus, one skilled in the art can empirically vary the inducer regimen to maximize the output of transgene expression for any given construct of interest. The optimal regimen will be based, in part, on potential toxicities of the desired protein to the producer cell line, the extent to which transcription factors are in limited concentration as they bind to amplified promoter regions in episomes encoding the desired protein, and other factors which may limit the inherent production capabilities of the producer cell line.

The invention also provides a kit for expressing a desired protein by regulating transcription of the 107/402-T antigen. The kit comprises a human cell and a first episome. The human cell can be any of the cells described above. The first episome comprises a papovavirus origin of replication, such as the SV40 or BK origins of replication, to which the 107/402-T antigen binds. The first episome is used as a vector for a coding sequence for the desired protein. The coding sequence for the desired protein can be inserted into the first episome using standard recombinant DNA techniques. The first episome can also contain an active promoter, for example the regulatory region from elongation factor-1α. A restriction enzyme site or multiple cloning site can be included in the first episome to permit incorporation of the protein coding sequence, or the first episome can be provided with a coding sequence for a desired protein already inserted.

The human cell also contains one or more copies of a first DNA sequence encoding an inducible transcriptional transregulator, a minimally active promoter, and a second DNA sequence encoding the 107/402-T antigen. The DNA sequences encoding the inducer transcriptional transregulator and the 107/402-T antigen can be integrated into the genome of the cells or can be on the first episome or a second episome.

Regulating Activity of the 107/402-T Antigen Using a Protein Switch

In another embodiment of the invention, replication of an expression vector encoding the protein to be expressed is controlled by regulating the activity of the 107/402-T antigen by means of a “protein switch.” This regulation is accomplished by providing a cell with a fusion protein comprising two protein segments fused together by means of a peptide bond. The first protein segment comprises the 107/402-T antigen. The second protein segment comprises a truncated hormone binding domain of a progesterone receptor. The truncated hormone binding domain binds only synthetic antiprogestins, such as RU486, and does not bind progesterone. The progesterone receptor is preferably a human progesterone receptor. A wild-type sequence of a human progesterone receptor is shown in SEQ ID NO:2. Preferred truncated hormone binding domains comprise amino acids 640-891 or amino acids 640-914 of a human progesterone receptor. Other segments of the human progesterone receptor have comparable properties (DeLort and Capecchi, 1996). Mutant progesterone receptors, which comprise amino acids not normally present in a progesterone receptor, also can be used. One sequence of a mutant receptor is taught in Vegeto et al. (1992). This particular mutant progesterone receptor lacks 54 authentic C-terminal amino acids and includes 12 novel amino acids at the C-terminal.

The truncated hormone binding domain is covalently bound at its amino terminus to amino acid 673 of the 107/402-T antigen. In the absence of antiprogestin, the progesterone receptor portion of the fusion protein interferes with the ability of the 107/402-T antigen to function as a replication transactivator. In the presence of an antiprogestin such as RU486, however, the conformation of the hormone binding domain of the progesterone receptor changes, and 107/402-T antigen becomes functional. Replication of an expression vector that contains a papovavirus origin of replication can then take place. Thus, the fusion protein functions as a protein switch, which regulates the replication activating activity of 107/402-T antigen.

A transcription cassette can be constructed comprising a coding sequence for the fusion protein. This coding sequence comprises a coding sequence for the 107/402-T antigen and a coding sequence for the truncated hormone binding domain of the progesterone receptor inserted directly 3′ of codon 673 of the 107/402-T antigen. The transcription cassette preferably includes codons 640-891 or codons 640-914 of a human progesterone receptor coding sequence. A transcription cassette also includes a promoter that controls transcription of the fusion protein coding sequence, an internal ribosome entry site, and a coding sequence for a protein whose expression is desired.

The polynucleotides or transcription cassettes described above can be included in an expression vector, which can be constructed using recombinant DNA techniques well-known in the art. The expression vector preferably comprises an active promoter for expressing large quantities of the fusion protein. A promoter such as the CMV immediate early promoter-enhancer, or a highly active human promoter such as the regulatory region from elongation factor-1α, can be used for this purpose. Tissue-specific promoters also can be used. As used herein, a “tissue-specific” promoter includes promoters that are more transcriptionally active in one tissue than in other tissues. Examples include cardiac-specific promoters (e.g., the α-MHC_{5.5} promoter, α-MHC₈₆ promoter, and human cardiac actin promoter), kidney-specific promoters (e.g., the renin promoter), brain-specific promoters (e.g., the aldolase C promoter and the tyrosine hydroxylase promoter), and vascular endothelium-specific promoters (e.g., the Et-1 promoter and von Willebrand factor promoter). Alternatively, promoters that are specifically active in tumor cells, for example oncofetal promoters such as the α-fetoprotein promoter (Huber et al., 1991) or CEA promoter (Osaki et al., 1994), can be used to regulate expression of the fusion protein. The expression vector can include a papovavirus origin of replication, such as an SV40 or BK origin of replication.

The polynucleotide sequence encoding the fusion protein can be incorporated into suitable viral and non-viral expression vectors to express recombinant proteins in vitro or in vivo. For example, control of replication of non-integrating viral vectors, such as herpes simplex and adenoviral vectors, would permit external control of vector replication, thereby boosting transgene expression. In a cancer gene therapy application, for example, such vectors could be used to kill cancer cells as a consequence of viral DNA replication. The fusion protein polynucleotide can be incorporated into a plasmid and introduced in vivo via a variety ofmethods, including direct injection as naked DNA, particle or spray bombardment, liposome/DNA complexes, or carrier/DNA complexes. The art is familiar with appropriate in vivo dosages of antiprogestins that can be administered to regulate activity of the fusion protein.

The promoter that regulates transcription of the DNA sequence encoding the fusion protein can also regulate transcription of the DNA sequence encoding the desired protein, for example, by including between the two coding sequences an internal ribosome entry site, as is known in the art. Alternatively, the expression vector can contain a separate promoter for regulating transcription of the DNA sequence encoding the desired protein.

Suitable mammalian cells for in vitro expression include primate cells (e.g., human cells and baboon, gorilla, chimpanzee, ape, and other simian cells), Chinese hamster ovary cells, and the like. For in vitro protein production, the host cell is grown in an appropriate culture medium. In a preferred embodiment, RU486 is added to the cell. Other antiprogestins, such as Onapristone, Org31710, or ZK112993, can also be used. The antiprogestin can be a component of the culture medium or can be added separately. The concentration of antiprogestin is selected by routine experimentation to result in an expression vector copy number for the particular cell line that results in maximal expression of the protein without cellular toxicity. Appropriate copy numbers, as measured, for example, by Southern blot (Cooper and Miron, 1993), range from at least 10 to at least 100, at least 100 to at least 1,000, at least 1,000 to at least 10,000, at least 10,000 to at least 50,000, at least 50,000 to at least 100,000, or at least 100,000 to at least 500,000 or more copies of the vector per cell. The concentration of antiprogestin which results in appropriate vector copy numbers for a particular cell type ranges from at least 1 nM to at least 10, 25, 50, 75, or 100 nM. The concentration of antiprogestin can be varied over time to achieve suitable copy numbers per cell.

The invention also provides a kit for expressing a desired protein by regulating activity of the 107/402-T antigen. In one embodiment, the kit comprises an expression vector that comprises a polynucleotide encoding a 107/402-T antigen-progesterone receptor fusion protein, as described above. In another embodiment, the kit comprises a mammalian cell, preferably a simian or human cell, that comprises the expression vector. If desired, the polynucleotide encoding the fusion protein can be integrated into the cell's genome. In each embodiment, expression of the fusion protein is controlled by an active promoter, as described above. The expression vector optionally comprises a papovavirus origin of replication to which the 107/402-T antigen binds, such as an SV40 or BK origin of replication. A coding sequence for the desired protein can be present in the expression vector. Alternatively, one or more restriction enzyme sites or a multiple cloning site can be included in the expression vector to permit incorporation of the protein coding sequence.

All patents and patent applications referenced herein are incorporated by reference. The following are provided for exemplification purposes only and are not intended to limit the scope of the invention that has been described in broad terms above.

### EXAMPLE 1

Construction of the 107/402-T Antigen Mutant

Wild-type SV40 large T antigen cDNA was isolated from plasmid pSG5-T as a 2.1 kb BamHI fragment. After XbaI linker addition, T antigen cDNA was ligated in the unique XbaI site of pRC/CMV (Invitrogen) to form pRC/CMV-T. In this vector, T antigen cDNA is transcriptionally controlled by the cytomegalovirus (CMV) immediate-early promoter. pRC/CMV contains an SV40 DNA origin; pRC/CMV-T therefore contains a complete SV40 replicon.

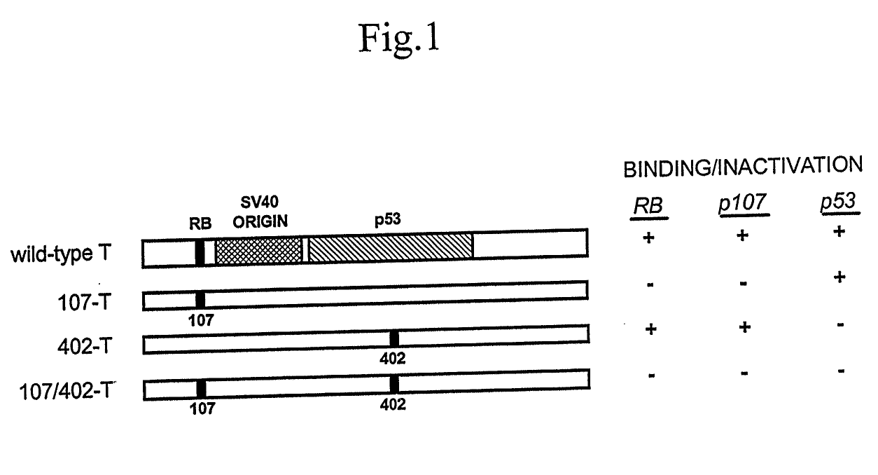

In a similar fashion, pRC/CMV.107-T was constructed from pSG5-K1, which encodes a mutant T antigen substituting lysine for glutamic acid at codon 107 (Kalderon and Smith, 1984). pRC/CMV.402-T and pRC/CMV.107/402-T were constructed by substituting a 1067 base pair HpaI C-terminal fragment of T antigen from pRC/CMV-T and pRC/CMV.107-T, respectively, with the corresponding T antigen fragment from a mutant SV40 virus clone that encodes a point mutation which substitutes glutamic acid for aspartic acid at codon 402 (clone 402DE) (Lin and Simmons, 1991). These point mutations are shown schematically in FIG. 1.

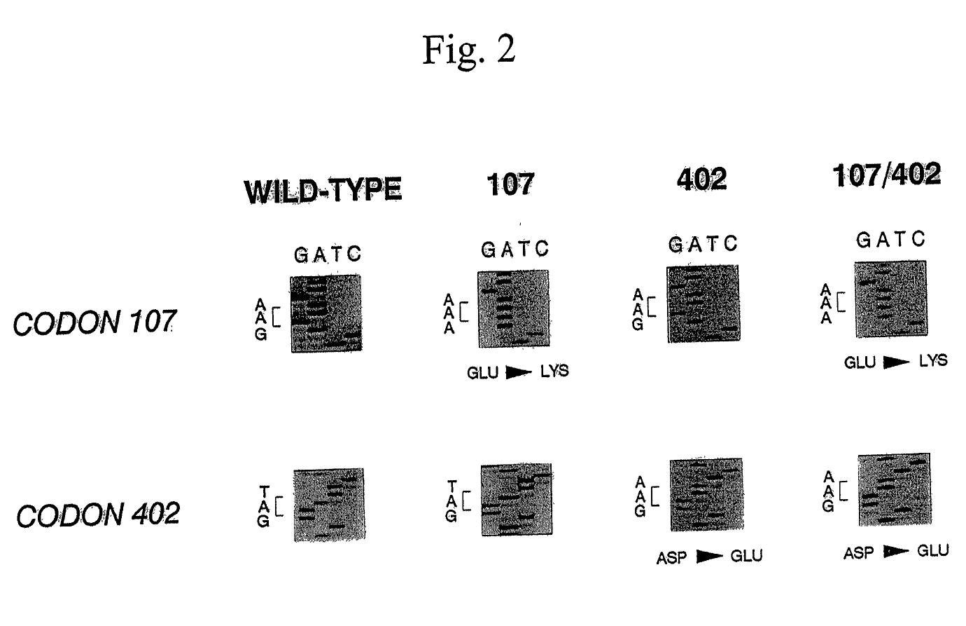

DNA sequence analysis confirmed in-frame ligation of the HpaI fragment, and also verified presence or absence of point mutations in codons 107 and 402 for each plasmid construct (FIG. 2).

### EXAMPLE 2

107/402-T Antigen Does Not Bind to Wild-type RB, p07, and p53 Proteins

The biochemical correlate of SV40 large T antigen-mediated induction of tumorigenicity is complex formation with p53, RB, and possibly RB-related proteins such as p107 (Linzer and Levine, 1979; DeCaprio et al., 1988; Ewen et al., 1991; Claudio et al., 1994). To evaluate directly the ability of 107/402-T to bind to wild-type RB, p107, and p53, in vitro translated wild-type and mutant T antigens were added to extracts from CV-1 cells in which human RB, p107, or p53 were transiently expressed at high levels.

Wild-type and mutant T antigens were translated in vitro in the presence of ³⁵S-methionine, using a reticulocyte lysate system as described by the manufacturer (Promega). Labeled T antigen (2×10⁵ dpm) was added to extracts from CV-1 cells transiently expressing human RB, p107, or p53 at high levels. CV-1 cells were infected with a vaccinia virus vector encoding T7 RNA polymerase. One hour later cells were transfected with derivatives of the pTM1 plasmid (Moss et al., 1990) containing a T7 polymerase site immediately upstream of either human RB, p107, or p53 cDNA.

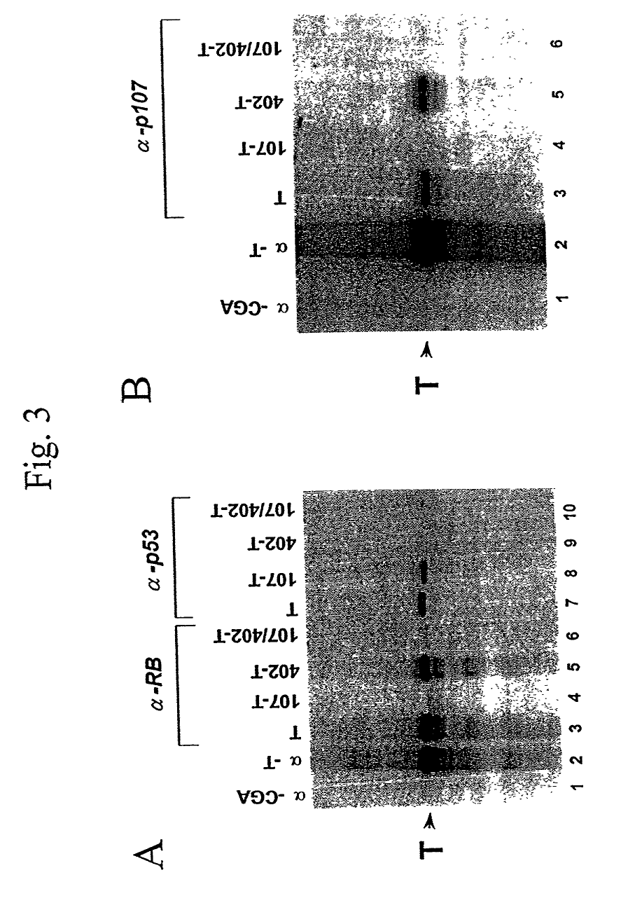

Approximately eighteen hours later, cells were harvested using a lysis buffer as described in Cooper et al. (1994). Immunoprecipitation analysis was performed using monoclonal antibodies to RB (clones G3-245, Pharmingen), p107 (clone SD9, Oncogene Science), and p53 (clone 1801, Oncogene Science), as described in DeCaprio et al., 1988. Band intensities were scanned using a phosphorimager to quantitate binding interactions. The results of these experiments are shown in FIGS. 3A and 3B.

As shown in Table I, little or no binding of 107/402-T was detected in these experiments, demonstrating that 107/402-T does not bind significantly to either RB, p107, or p53.

TABLE 1
Binding of wild-type and mutant SV40 large T antigens to RB
p107, and p53 tumor suppressor gene products

| Tumor suppressor | Observed signal compared to T | | | |
|---|---|---|---|---|
| gene product | T, % | 107-T, % | 402-T, % | 107/402-T, % |
| RB | 100 | 0.03 | 67 | 0.07 |
| p107 | 100 | 0 | 79 | 0 |
| p53 | 100 | 36.2 | 0 | 0 |

### EXAMPLE 3

107/402-T is Replication-competent and is a More Effective Replication Activator Than Wild-type Large T Antigen

The replication activities of wild-type and mutant SV40 large T antigens were evaluated in a panel of human cell lines, including HT-1376 (bladder carcinoma), 5637 (bladder carcinoma), MCF-7 (breast carcinoma), SW480 (colon cancer), Hs68 (fibroblast), HepG2 (hepatoma), and RAJI (lymphoma).

Cells were transfected using either lipofectin (GIBCO) (Cooper and Miron, 1993), calcium phosphate DNA precipitation (Graham and Van der Eb, 1973), or electroporation. Specific transfection conditions were optimized to achieve a transfection efficiency of at least 1% while minimizing cell toxicity. The day after gene transfer, cell cultures were split to maintain log phase growth for the duration of the experiment.

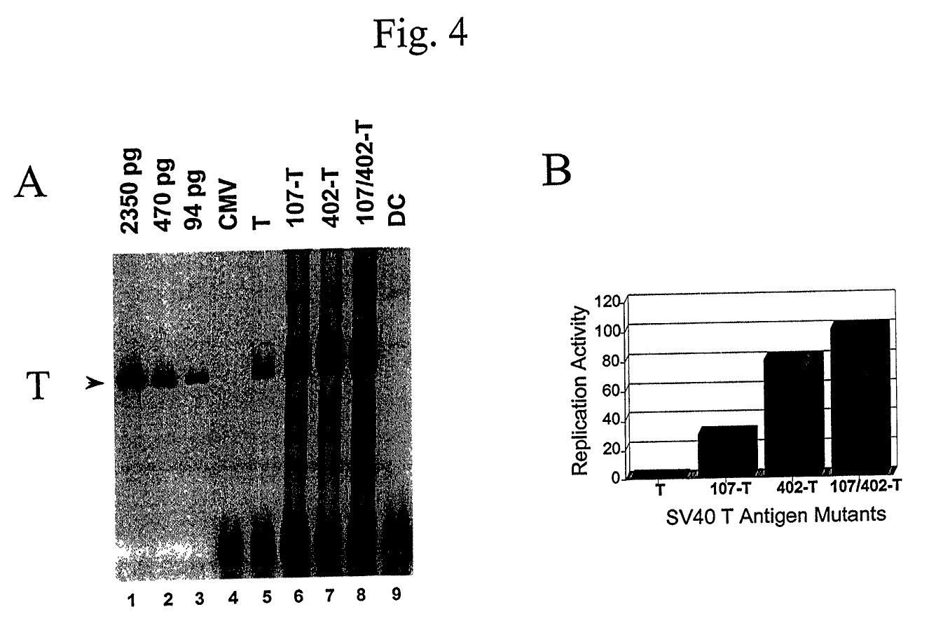

DNA harvested from transient transfectants was evaluated for the presence of extrachromosomal plasmid replication by resistance to DpnI digestion, as described in Cooper and Miron (1993). As shown in FIG. 4A, significant replication activity was observed in human cells. In HepG2 cells, for example, a copy number of approximately 25,000 per cell was noted by two days post-gene transfer, and copy numbers ranging from 80 to 100,000 were observed in other human cell types (Cooper et al. 1997). Furthermore, in the HepG2 cell line the replication activating ability of 107/402-T was increased over that of wild-type SV40 large T antigen by a factor of one hundred (FIGS. 4A and 4B).

### EXAMPLE 4

107/402-T has Enhanced Replication Activity Compared to Wild-type T Antigen During S-phase of the Cell Cycle

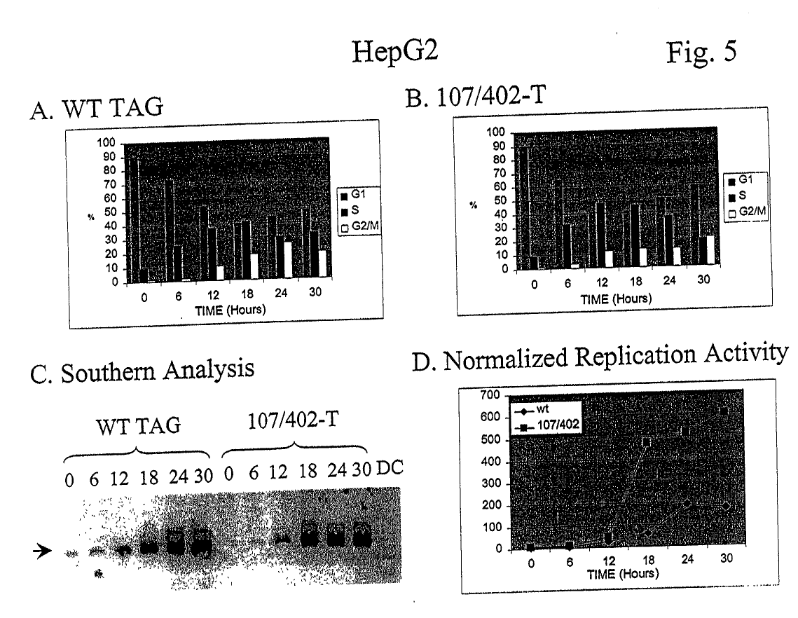

As described in Example 3, the copy number of pRC/CMV.107/402-T in HepG2 human hepatoma cells was 100-fold higher than pRC/CMV.T at 2 days post gene transfer. To further investigate the mechanism underlying this difference in episomal copy number, the cell cycle dependence of replication activity was evaluated. HepG2 cells were transfected with pRC/CMV.107/402-T or pRC/CMV.T. Twenty-four hours later, cells in early G1 of the cell cycle were isolated by centrifugal elutriation. The initial population of G1-enriched cells (time 0) and cells 6, 12, 18, 24, and 30 hours after replating were assayed for cell cycle analysis (FACS analysis of propidium iodide-stained cells, FIGS. 5A and 5B) and episomal copy number (Southern blot analysis, FIG. 5C). Log-phase growth conditions were maintained during replating. The band intensities in FIG. 5C were normalized for transient transfection efficiency by FACS analysis of T antigen expression. The normalized replication activity is presented in FIG. 5D.

The cell cycle analysis demonstrated that the peak time period for traversal of S phase was 12-18 hours post-replating. By 30 hours post-replating, cells transfected with pRC/CMV.107/402-T had a 3.4-fold increase in copy number compared to pRC/CMV.T. The increase in replication activity appeared to be largely restricted to S-phase of the cell cycle and accounts for the enhanced replication activity of 107/402-T in comparison to wild-type T antigen.

### EXAMPLE 5

107/402-T Significantly Enhances Gene Expression Compared to Wild-type T Antigen

To evaluate replication reporter transgene expression mediated by 107/402-T or wild-type T antigen, HepG2 hepatoma cells were co-transfected with pCMVSEAP (CMV immediately-early promoter transcribing secreted alkaline phosphatase) and either pRSVwt-T, pRSV.107/402-T, or pRSV (no insert). These RSV expression vectors lack an SV40 DNA origin and hence will not replicate in transiently transfected cells; the duration of T antigen expression will therefore be limited. In contrast, pCMVSEAP contains an SV40 DNA origin and will replicate extrachromosomally in cells co-expressing T antigen. HepG2 cells in 100 mm dishes were cotransfected with 10 ng of pCMVSEAP and 14 μg of the RSV-based vectors.

On day one, samples of medium from the cells were saved and the cells were trypsinized and replated. At each 24 hour interval, media was harvested and cell extracts were prepared to calculate the total amount of protein per well. Alkaline phosphatase activity was measured in media using a commercial chemiluminescent assay (Tropix).

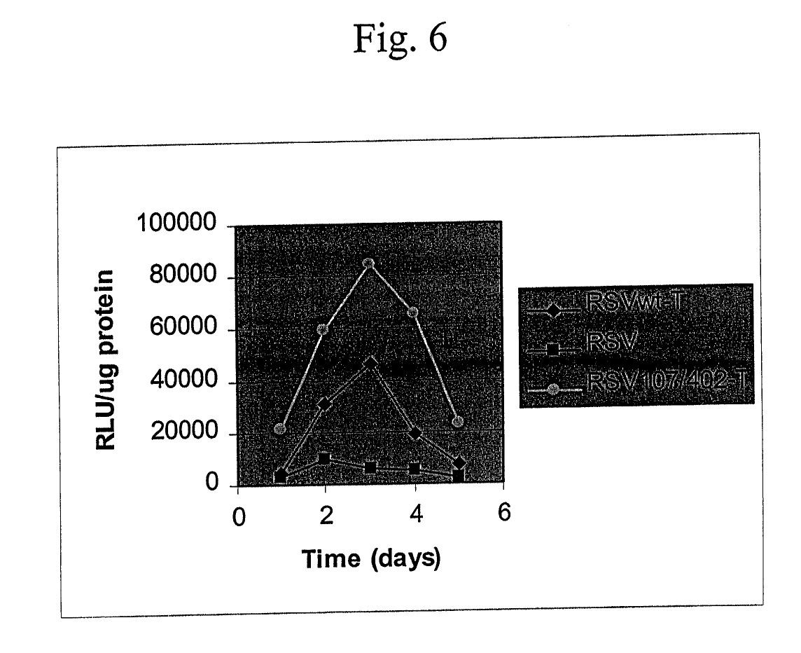

Data are presented in FIG. 6 as relative light units per μg of protein per 24 hours. A two- to five-fold improvement in alkaline phosphatase activity was observed in the 107/402-T co-transfectants compared to the wild-type T antigen co-transfectants. The level of alkaline phosphatase activity in the 107/402-T co-transfectants was greater than an order of magnitude higher than the pRSV co-transfectants (non-replicating standard expression vector control), emphasizing the importance of this replicating expression system for producing high levels of recombinant protein.

### EXAMPLE 6

Gene-modified Cells can be Prepared to Express 107/402-T under Transcriptional Control of the Tetracycline-controlled Gene Switch

To prepare a human cell line in which expression of 107/402-T would be under control of doxycycline, HT-1376 human bladder carcinoma cells were sequentially transfected with three plasmid constructs: (a) pTET-OFF, which encodes the tetracycline-controlled transcriptional transactivator (tTA) under control of the CMV immediate-early promoter and the neomycin resistance gene under control of the SV40 early promoter, (b) pTRE.107/402-T, which encodes 107/402-T under control of the CMV minimal promoter and contains the tetracycline operon (binding site of tTA just upstream of the CMV minimal promoter), and (c) pCMVhygro, which encodes the hygromycin resistance gene under control of the CMV promoter. HT-1376 cells were first transfected with pTET-OFF, and neomycin resistant clones of stable transfectants were characterized by transiently transfecting clones with pTRE.luciferase in the presence or absence of doxycycline. Clones which yielded significant luciferase activity only in the absence of doxycycline (but no detectable luciferase activity in the presence of doxycycline) were then co-transfected with pTRE.107/402-T and pCMVhygro. Again, single cell clones of stable transfectants were screened for high basal levels of 107/402-T and complete turn-off of 107/402-T expression in the presence of doxycycline.

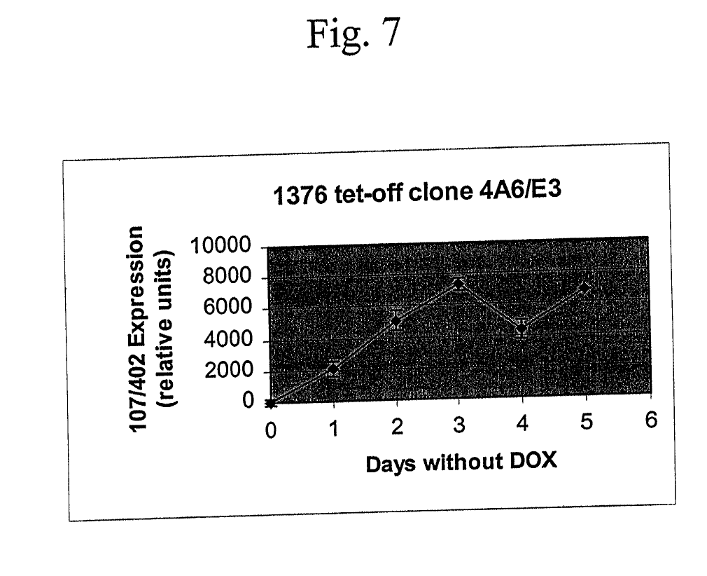

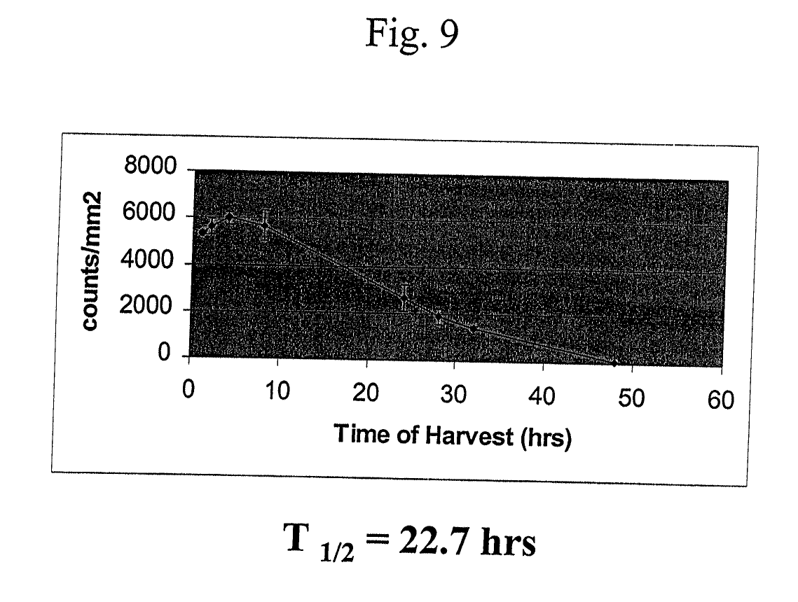

An example of an HT-1376 tet-off clone that demonstrates precise control of 107/402-T expression is shown in FIG. 7. FIG. 7 shows the time course of induction of 107/402-T expression upon washout of saturating amounts of doxycycline (3 ng/ml). Steady-state levels of 107/402-T are achieved by 3 days. The doxycycline concentration-dependence of 107/402-T expression is presented in FIG. 8. The half-maximal inhibitory concentration of doxycycline is approximately 0.01 ng/ml. The half-life of 107/402-T expression after addition of 3 ng/ml doxycycline is presented in FIG. 9. The observed decrease of 107/402-T expression yields a half-life of 22.7 hours in this cell line.

The data in FIGS. 7-9 permit design of a cyclic regimen of doxycyline that will fluctuate levels of 107/402-T expression about a predetermined level of 107/402-T expression. Such a cyclic profile of 107/402-T expression will, in turn, generate sustained and elevated levels of transgene expression derived from a replicating reporter plasmid encoding the SV40 DNA origin.

### EXAMPLE 7

Cyclic Regimens of Doxycycline can be Used to Control Transgene Expression in the Gene-modified Cells of Example 6

The HT-1376 clone described in Example 6 (clone 4A6/E3) was transfected with pCMVSEAP, an expression plasmid in which the CMV immediate-early promoter regulates transcription of a secreted alkaline phosphatase reporter gene. pCMVSEAP contains the SV40 DNA origin and hence will replicate extrachromosomally in the presence of 107/402-T antigen. In this experiment, cells were incubated without doxycycline for 4 days to produce maximal levels of 107/402-T antigen expression. Duplicate dishes of cells (A, B) were then transfected with pCMVSEAP (day 0) and replated in a series of 60 mm wells for analysis of alkaline phosphatase expression at a series of time points. Doxycycline (50 ng/ml) was added back to the cells between days 2-5 to block production of 107/402-T antigen. Media was changed every 24 hours to determine daily alkaline phosphatase activity.

To measure secreted alkaline phosphatase activity, 25 μl of media were assayed using a chemiluminescent assay as described by the manufacturer (Tropix, Inc.). The light units per well were then calculated, and cells were harvested for protein determination. Activity is expressed as relative light units of alkaline phosphatase activity per μg of protein per 24 hours.

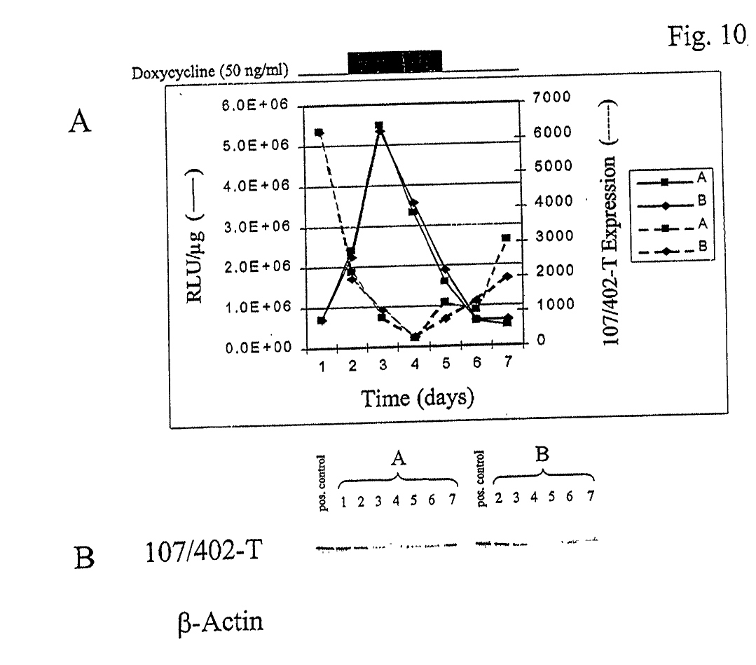

As shown in FIG. 10A, alkaline phosphatase activity peaks on day 3 and then declines. In FIG. 10B, the protein extracts were evaluated for 107/402-T expression by Western blot analysis. The same blot was reprobed for β-actin to ensure equal loading of extracts per well. Normalized levels of 107/402-T antigen expression are plotted in FIG. 10A and demonstrate that levels of 107/402-T antigen can be cycled by appropriate exposure of the cells to doxycycline.

These data demonstrate the ability to cycle levels of transgene expression using the method of the invention. Furthermore, levels of transgene expression can be optimized for a given application by simply altering the regimen of doxycycline exposure to yield appropriate levels of episomal amplification. This modular and flexible system permits optimization of expression for a given transgene based on potential toxicities of the transgene to the host production cell as well as the inherent synthetic capabilities of the producer cell.

### EXAMPLE 8

Construction and Replication Competency of 107/402-T/PR Fusion Proteins

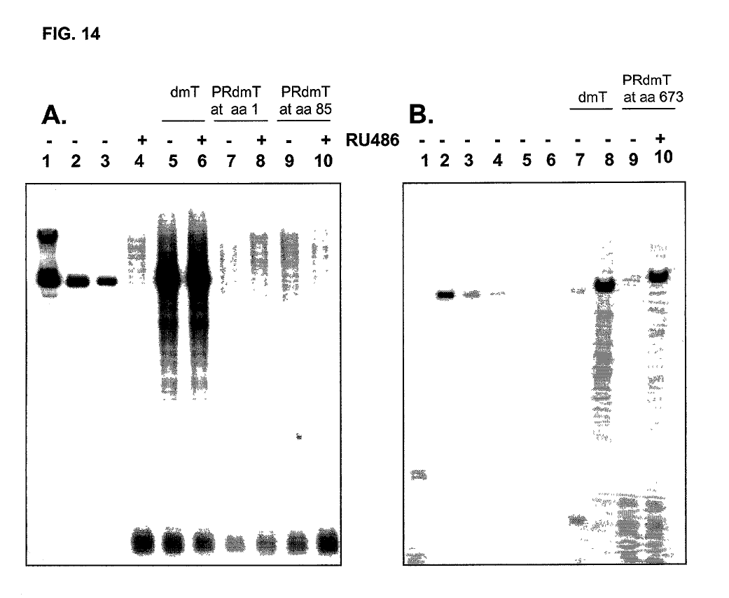

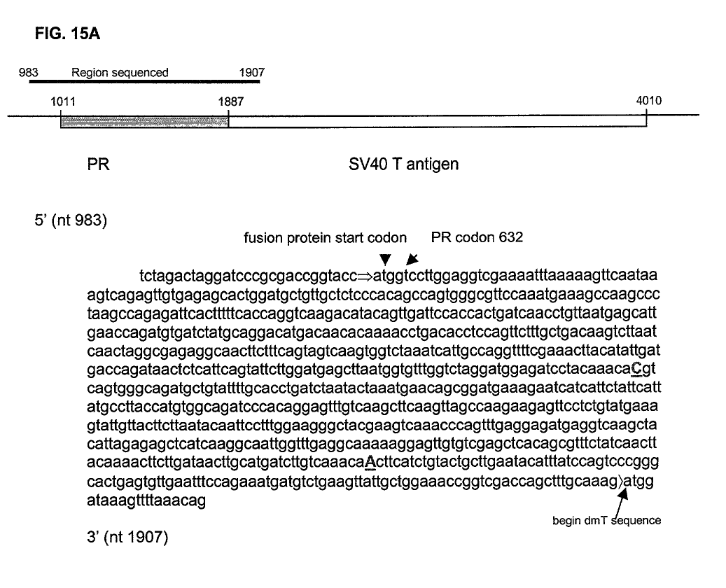

Constructions were made that inserted a truncated progesterone receptor (PR) hormone binding domain fragment (made from a region encompassing approximately PR amino acids 630-915, depending upon cloning strategy) into the double mutant 107/402-T antigen (dmT) open reading frame at amino acids 1, 85, or 673 of the dmT sequence. Following the construction of these fusions, Southern blot assays were performed to test the replication competency of the new constructs. A replication-competent dmt/PR fusion protein will transactivate replication of plasmids containing the SV40 DNA origin. Due to differences in nucleotide methylation patterns in bacteria and human cells, plasmid DNA prepared in bacteria will be digested by DpnI whereas DNA amplified in human cells will be resistant to DpnI digestion. Therefore, Southern analysis of DNA from transfected cells can distinguish between the input DNA used for gene transfer and any newly replicated plasmid; bands resistant to DpnI digestion represent newly amplified plasmid DNA. Furthermore, these Southern blots test the function of the PR portion of the fusion protein by assaying replication in the presence or absence of RU486, a progesterone receptor antagonist. The results are shown in FIGS. 14A and B.

In one experiment, 100 mm dishes of HepG2 cells (plated the day before transfection at 8×10⁶ cells per plate) were transfected with the above plasmids using calcium phosphate transfection technique and 100 μM chloroquine. Samples which were to be treated with RU486 were brought to 1 μM RU486 (Sigma) when transfection media was replaced with complete media (˜4 hrs post transfection). RU486 was not refreshed after that point. Transfections were harvested at about 48 hours post-transfection. DNAs were isolated using a QiaAmp Blood Genomic DNA isolation kit (Qiagen). DNAs were quantified by A₂₆₀ readings, and 0.5 μg samples were loaded per lane. All samples except standard curve positive controls (lanes 1-3 of FIG. 14A) were digested with Dpn I and Apa I restriction enzymes prior to loading on the gel. The DNA in the gel was transferred to a nitrocellulose membrane by wicking. The blot was probed using a fragment of the vector pRcCMV.

The results of this experiment are shown in FIG. 14A (lane 1, 2160 pg positive control wild type T plasmid, pRcCMVT; lane 2, 713 pg positive control wild type T plasmid, pRcCMVT; lane 3, 216 pg positive control wild type T plasmid, pRcCMVT; lane 4, RU486-stimulated negative control empty vector pRcCMV; lane 5, unstimulated dmT plasmid pRcCMV/dmT; lane 6, RU486-stimulated 107/402-T plasmid pRcCMV/dmT; lane 7, unstimulated PRdmT fusion protein with PR insertion at amino terminus of dmT: pRcCMV1/PR632-914/dmT; lane 8, RU486-stimulated PRdmT fusion protein with PR insertion at amino terminus of dmT: pRcCMV1/PR632-914/dmT; lane 9, unstimulated PRdmT fusion protein with PR insertion at amino acid 85 of dmT: pRcCMV85/PR631-915/dmT; lane 10, RU486-stimulated PRdmT fusion protein with PR insertion at amino acid 85 of dmT: pRcCMV85/PR631-915/dmT).

In another experiment, 100 mm dishes of HepG2 cells (plated the day before transfection at 10×10⁶ cells per plate) were transfected with the above plasmids using LipofectAMINE (Gibco) transfection. Dishes that were to be stimulated with RU486 were brought to 1 μM RU486 (Sigma) final concentration when transfection media was changed to complete media (˜4 hrs post transfection). RU486 was not refreshed after that time. Transfections were harvested at about 72 hours post-transfection. DNAs were isolated using QiaAmp Blood Genomic DNA isolation kit (Qiagen). DNAs were quantified by A₂₆₀ readings, and 5 μg samples were loaded per lane. All experimental samples (lanes 1, 5-10 of FIG. 14B) were digested with DpnI and Apa LI restriction enzyme prior to loading on an agarose gel. The DNA in the gel was transferred to a nitrocellulose membrane by wicking. The blot was probed using a 2384 bp XbaI-Spe fragment of the vector pKCPIR that had been labeled using an Amersham ECL system for nonradioactive nucleotide labeling. The gel was developed using Pierce SuperSignal West Dura ECL substrate kit and imaged using a BioRad MultiImager.

The results of this experiment are shown in FIG. 14B ((lane 1, negative control genomic DNA spiked with pKCPIR1ucBGH5.3−; lane 2, 250 pg positive control pKCPIR1ucBGH5.3−; lane 3, 100 pg positive control pKCPIR1ucBGH5.3−; lane 4, 25 pg positive control pKCPIR1ucBGH5.3−; lane 5, negative control genomic DNA; lane 6, irrelevant plasmid pKCPIR1ucBGH5.3−; lane 7, 107/402-T without SV40 DNA origin of replication: pKCPIRdmT5.9−; lane 8, 107/402-T with SV40 DNA origin of replication: pKCPIRdmT6.2+; lane 9, unstimulated fusion protein with PR insertion at amino acid 673 of dmT and containing an SV40 DNA origin of replication: pKCPIR673/PR640-914/dmT+; lane 10, RU486-stimulated fusion protein with PR insertion at amino acid 673 of dmT and containing an SV40 DNA origin of replication: pKCPIR673/PR640-914/dmT+).

As seen in FIGS. 14A and B, neither the construction with insertion at amino acid 1 nor the insertion at amino acid 85 were capable of replication in mammalian cells. The fusion protein with the PR insertion at amino acid 673 was able not only to replicate, but also to do so in an RU486-dependent manner.

### EXAMPLE 9

Sequencing of 107/402-T/PR Fusion Constructs

Due to the unexpected results of the Southern blot experiments (i.e., only one successful fusion protein as detected by Southern), the three constructs were sequenced. Sequencing results would allow the distinction between unsuccessful fusion proteins and failures due to cloning error. Fusion proteins with PR insertion into dmT at amino acid 1 or 85 were sequenced in the region of cloning junction. Fusion protein with PR insertion into dmT at amino acid 673 was sequenced in its entirety.

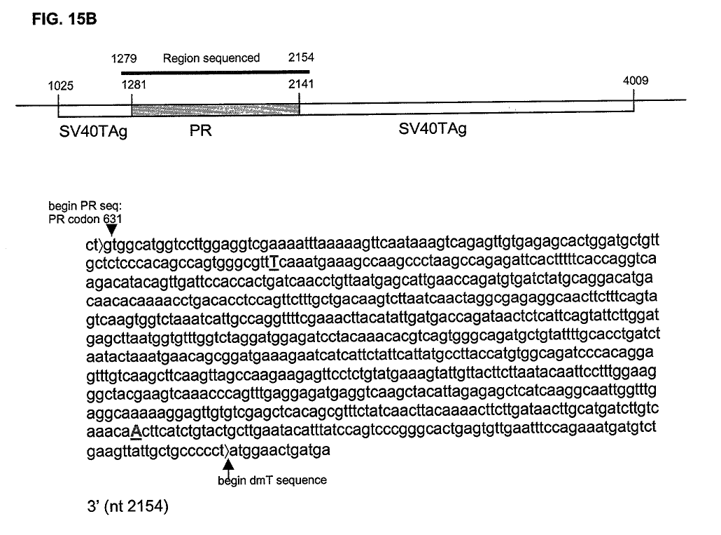

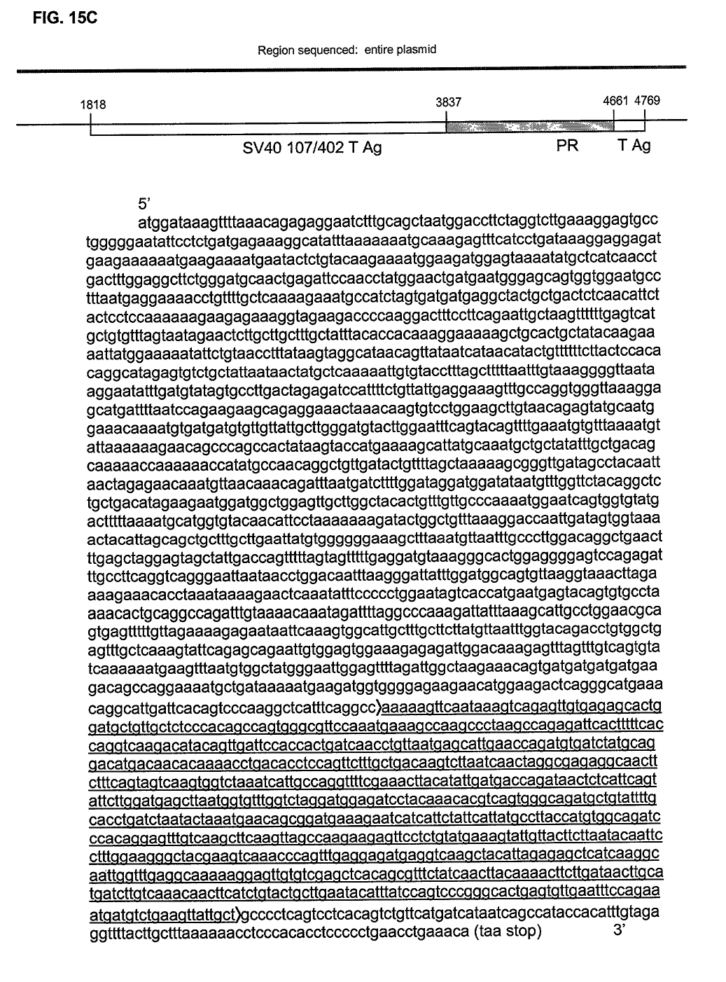

Sequencing of pRcCMV1/PR632-914/dmT (insertion at amino acid 1; FIG. 15A; SEQ ID NO:3) and pRcCMV85/PR631-915/dmT (insertion at amino acid 85; FIG. 15B; SEQ ID NO:4) were performed by Lark Technologies, Inc. Sequencing of pKCPIR673/PR640-914/dmT+ (insertion at amino acid 673; FIG. 15C; SEQ ID NO:5) was performed by Seqwright. The results are shown in FIGS. 15A-C. Letters in bold, capitalized, and underlined represent unexpected nucleotides when compared with predicted sequence. These changes are not within the joining region of cloning.

Sequencing of the PRdmT construct with the PR fragment insertion at amino acid 1 of the dmT sequence identified two nucleotide changes. Sequencing of these constructs found two conservative nucleotide changes in the PRdmT construct with the PR fragment insertion at the amino terminus of dmT. Both nucleotide changes were conservative and, therefore, the expected amino acids were encoded. Sequencing does not explain the lack of replication of this construct.

Sequencing of the PRdmT construct with the PR fragment insertion at amino acid 85 of the dmT sequence identified two nucleotide changes. One of these changes was conservative, and the other was not. This nonconservative change altered proline 663 to a valine in the final protein. However, this amino acid is located in the “hinge” region (641-686) of the PR protein rather than the hormone binding domain (687-933). We would not predict this amino acid change to alter the function of the fusion protein. Therefore, sequencing of this construct did not explain the lack of replication as identified by Southern blot.

The open reading frame of PRdmT fusion protein with the PR insertion at amino acid 673 (pKCPIRdmT673PR640-914+) was found to align completely with the predicted sequence. This was expected, because this construct was replication-competent as detected by Southern blot and also displayed RU486 sensitivity.

### EXAMPLE 10

In vitro Translation

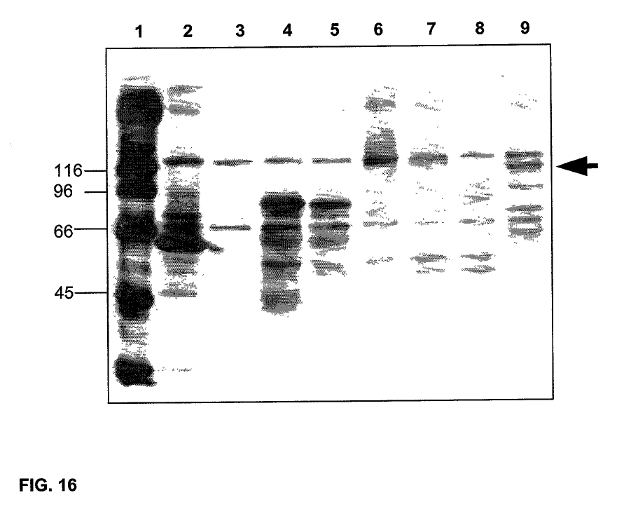

To evaluate the ability of the fusion constructs described above to produce fusion proteins, in vitro translation assays were performed. The three PRdmT constructs should produce a unique protein band on SDS-PAGE that should be larger in molecular weight than dmT alone. As seen in FIG. 16, however, only the plasmid with PR insertion at amino acid 673 of dmT produced such a unique protein band.

One microgram DNA samples were transcribed and translated using a TNT/T7 kit (Promega) and a Transcend tRNA Biotinylated Detection System (Promega) that incorporates biotinylated lysines into transcribed proteins. The IVT samples were run on an 8% acrylamide gel. SDS-PAGE was run at 85 V until complete. The gel was then transferred to a nitrocellulose membrane using a semi-dry transfer apparatus running at 150 mA for 1 hour. The nitrocellulose membrane was probed for 1 hour at room temperature with a streptavidin/HRP conjugate (Promega) at 1:1000 dilution. The streptavidin binds the biotinylated lysines in the proteins on the nitrocellulose. The location of such binding reactions can then be detected using the HRP enzyme portion of the bound conjugate. After washing the blot, the HRP enzyme activity was detected using an ECL detection system. The blot was imaged for a 10 minute capture period using a BioRad MultiImager.

The results are shown in FIG. 16 (lane 1, molecular weight marker; lane 2, T7 luciferase positive control; lane 3, no DNA negative control; lane 4, wild type T antigen plasmid pRcCMVT; lane 5, double mutant (107/402) T plasmid pRcCMV/dmT; lane 6, wild type T fusion protein with progesterone receptor insertion at amino acid 85 (pRcCMV85/PR631-915/T) of T antigen sequence; lane 7, double mutant T fusion protein with PR insertion at amino acid 85 (pRcCMV85/PR631-915/dmT) of dmT sequence; lane 8, double mutant T fusion protein with PR insertion at amino acid 1 of dmT sequence (pRcCMV1/PR632-914/dmT); lane 9, double mutant T fusion protein with PR insertion at amino acid 673 of dmT sequence (pRcCMV673/PR640-916/dmT)). A fusion PRdmT protein is found only in lane 9, indicating that the codon 673 insertion site in dmT uniquely produces fusion protein expression. The fusion protein is approximately 113 kD, as predicted by the encoded amino acid sequence (see arrow in FIG. 16).

### EXAMPLE 11

Activation of 107/402-T/PR Fusion Proteins by RU486

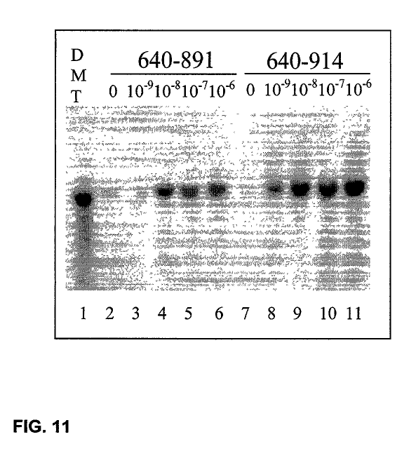

Different regimens of RU486 were used to examine the replication activity of two different fusion gene constructs incorporating codons 640-891 or 640-914 of the human progesterone receptor inserted at codon 673 of 107/402-T (FIG. 11). DNA sequence analysis confirmed construction of each fusion gene. Cells were grown in the presence or absence of various concentrations of RU486. On day 3 after transfection, cells were harvested for total cellular DNA, and samples were evaluated for extrachromosomal replication by Southern blot analysis. In FIG. 11, “DMT” refers to control “double-mutant” 107/402-T expression vector that defines 100% replication activity.

The preferred construct incorporates the PR 640-914 fragment. This construct has no observed replication activity at 3 days post-gene transfer in the absence of RU486, and significant activation is noted at an RU486 concentration of 1 nM. The 50% activation concentration of RU486 for the 640-914 construct is approximately 4 nM. Clinical peak serum levels of oral RU486 are typically in the 2-6 μM range, well above the concentration required to achieve activation of 107/402-T/PR640-914. In other studies, we have observed no replication of the 640-914 construct after six days of culture of transfected HepG2 cells in the absence of RU486.

### EXAMPLE 12

The 107/402-T/PR640-914 Switch is not Activated by Endogenous Steroid Hormones

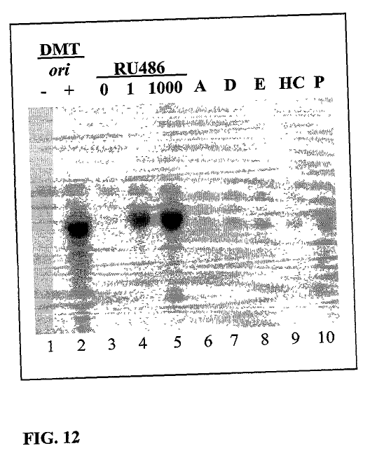

To determine if endogenous steroid hormones activate 107/402-T/PR640-914, HepG2 cells were transfected and incubated for three days in the presence of aldosterone, estradiol, hydrocortisone, or progesterone. Additionally, cells were incubated in the presence of the glucocorticoids agonist, dexamethasone. A Southern blot replication assay was performed. The results are shown in FIG. 12.

Cells were incubated in either the absence (lane 3) or presence (1 nM, lane 4; 1000 nM, lane 5) of RU486, or in the presence of 1 μM aldosterone (A), dexamethasone (D), estradiol (E), hydrocortisone (HC), or progesterone (P). Except for hydrocortisone, the 1 μM concentration level is approximately 500 to 1000-fold higher than physiologic concentrations of these hormones in humans. As a negative control, cells were transfected with a 107/402-T expression construct (DMT) lacking the SV40 DNA origin of replication (lane 1). Positive control cells (lane 2) were transfected with the complete 107/402-T replicon. No significant activation was noted for any endogenous steroid hormone.

### EXAMPLE 13

Design of an Externally-controlled Switch Vector

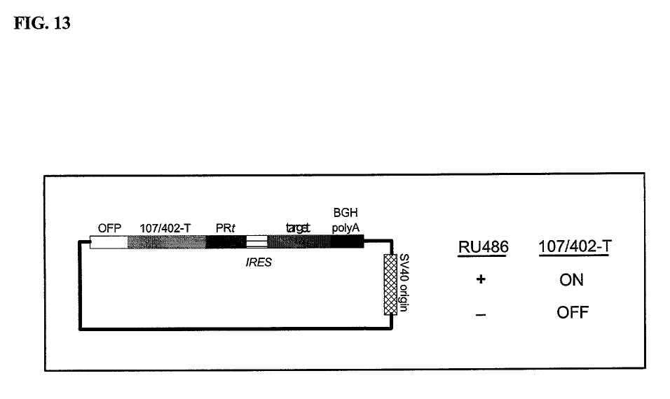

This fusion protein replication switch can be incorporated into expression vectors using a simple design that utilizes a single transcription cassette. As illustrated in FIG. 13, a therapeutic gene and 107/402-T/PR640-914 can be included in a single transcriptional cassette utilizing an internal ribosome entry site. The promoter in this vector can have tissue-specific and/or oncofetal characteristics to restrict gene expression and vector amplification to cancer cells. In FIG. 13, an oncofetal promoter (OFP) is exemplified.

REFERENCES

Boshart et al.(1985) Cell 41, 521-30

Caras and Weddell (1989) Science 243, 1196-98

Chen et al. (1990) J. Virol. 64, 3350-57

Chen et al. (1992) Oncogene 7, 1167-75

Christian et al. (1987) Cancer Res. 47, 6066-73

Claudio et al. (1994) Cancer Res. 54, 5556-60

Cooper et al. (1994) Oncol. Res. 6, 569-79

Cooper et al. (1997) Proc. Natl. Acad. Sci. U.S.A. 94, 6450-55

Cooper and Miron (1993) Human Gene Ther. 4, 557-66

DeLort and Capecchi (1996) Human Gene Therapy 7, 809-20

DeCaprio et al. (1988) Cell 54, 275-83

Deyerle et al. (1989) J. Virol. 63, 356-65

Ewen et al. (1991) Cell 66, 1155-64

Fiers et al. (1978) Nature 273, 113-20

Freshney, ed. (1986) ANIMAL CELL CULTURE

Gerard and Gluzman (1985) Mol. Cell. Biol. 5, 3231-40

Glover, ed. (1985) DNA CLONING: A PRACTICAL APPROACH, vols. 1 and 2

Gossen and Bujard (1992) Proc. Natl. Acad. Sci. U.S.A. 89, 5547-51

Gossen (1995) Science 268, 1766-69

Guo et al. (1996) Gene Ther. 3, 802-10

Hanahan et al. (1989) Science 246 1265-75

Huber et al. (1991) Proc. Natl. Acad. Sci. U.S.A. 88 8039-43

Kalderon and Smith (1984) Virol. 139, 109-37

Lin and Simmons (1991) J. Virol. 65, 2066-72

Linzer and Levin (1979) Cell 17, 43-52

Michalovitz et al. (1987) J. Virol 61, 2648-54

Moss et al. (1990) Nature 348, 91-92

Nordeen (1988) BioTechniques 6, 454-48

Osaki et al. (1994) Cancer Res. 54, 5258-61

Perbal (1984) A PRACTICAL GUIDE TO MOLECULAR CLONING

Reddy et al. (1978) Science 200, 494-502

Rivera et al. (1996) Nature Med. 2, 1028-32

Roberts et al. (1986) Cell 46, 741-52

Sambrook et al. (1989) MOLECULAR CLONING: A LABORATORY MANUAL

Shin et al. (1975) Proc. Natl. Acad. Sci. U.S.A. 72, 4435-39

Subramanian et al. (1977) J. Biol. Chem. 252, 355-67

Van Heuverswyn et al. (1978) Eur. J. Biochem. 100, 51-60

Vegeto et al. (1992) Cell 69, 703-13

Yang et al. (1979) Science 206, 456-61

Walter and Blobel (1982) Nature 299, 691-98

Wang et al. (1997) Gene Ther. 4, 432-41

1. A fusion protein, comprising:
a 107/402-T antigen; and
a truncated hormone binding domain of a progesterone receptor, wherein the truncated hormone binding domain binds an antiprogestin but does not bind progesterone, wherein the truncated hormone binding domain is covalently bound at its amino terminus to amino acid 673 of the 107/402-T antigen.

2. The fusion protein of claim 1 wherein the antiprogestin is RU486.

3. The fusion protein of claim 1 wherein the truncated hormone binding domain comprises amino acids 640-891 of a human progesterone receptor.

4. The fusion protein of claim 1 wherein the truncated hormone binding domain consists of amino acids 640-891 of a human progesterone receptor.

5. The fusion protein of claim 1 wherein the truncated hormone binding domain comprises amino acids 640-914 of a human progesterone receptor.

6. The fusion protein of claim 1 wherein the truncated hormone binding domain consists of amino acids 640-914 of a human progesterone receptor.

7. An isolated polynucleotide, comprising:
a first coding sequence for a 107/402-T antigen; and
a second coding sequence for a truncated hormone binding domain of a progesterone receptor, wherein the truncated portion of the hormone binding domain binds an antiprogestin but does not bind progesterone, wherein the 5′-most codon of the second coding sequence is located directly 3′ of codon 673 of the first coding sequence.

8. The polynucleotide of claim 7 wherein the antiprogestin is RU486.

9. The polynucleotide of claim 7 wherein the second coding sequence comprises codons 640-891 of a human progesterone receptor coding sequence.

10. The polynucleotide of claim 7 wherein the second coding sequence consists of codons 640-891 of a human progesterone receptor coding sequence.

11. The polynucleotide of claim 7 wherein the second coding sequence comprises codons 640-914 of a human progesterone receptor coding sequence.

12. The polynucleotide of claim 7 wherein the second coding sequence consists of codons 640-914 of a human progesterone receptor coding sequence.

13. The polynucleotide of claim 7 further comprising a promoter that regulates transcription of the first and second coding sequences.

14. The polynucleotide of claim 13 wherein the promoter is an oncofetal promoter.

15. The polynucleotide of claim 13 wherein the promoter is a tissue-specific promoter.

16. The polynucleotide of claim 7 further comprising an internal ribosome entry site.

17. The polynucleotide of claim 16 further comprising a restriction enzyme site for insertion of a third coding sequence for a desired protein.

18. The polynucleotide of claim 17 further comprising the third coding sequence.

19. A transcription cassette comprising:
a first coding sequence for a 107/402-T antigen;
a second coding sequence comprising codons 640-914 of a human progesterone receptor coding sequence, wherein the 5′-most codon of the second coding sequence is located directly 3′ of codon 673 of the first coding sequence;
a promoter that controls transcription of the first and second coding sequences;
an internal ribosome entry site; and
a third coding sequence for a desired protein.

20. An expression vector comprising a polynucleotide encoding a fusion protein, wherein the polynucleotide comprises:
a first coding sequence for a 107/402-T antigen; and
a second coding sequence for a truncated hormone binding domain of a progesterone receptor, wherein the truncated hormone binding domain binds an antiprogestin but does not bind progesterone, and wherein the 5′-most codon of the second coding sequence is located directly 3′ of codon 673 of the first coding sequence.

21. The expression vector of claim 20 wherein the antiprogestin is RU486.

22. The expression vector of claim 20 wherein the second coding sequence comprises codons 640-891 of a human progesterone receptor coding sequence.

23. The expression vector of claim 20 wherein the second coding sequence consists of codons 640-891 of a human progesterone receptor coding sequence.

24. The expression vector of claim 20 wherein the second coding sequence comprises codons 640-914 of a human progesterone receptor coding sequence.

25. The expression vector of claim 20 wherein the second coding sequence consists of codons 640-914 of a human progesterone receptor coding sequence.

26. The expression vector of claim 20 further comprising a first promoter that regulates transcription of the polynucleotide.

27. The expression vector of claim 26 wherein the first promoter is an oncofetal promoter.

28. The expression vector of claim 26 wherein the first promoter is a tissue-specific promoter.

29. The expression vector of claim 20 wherein the polynucleotide further comprises:
an internal ribosome entry site; and
a restriction enzyme site for insertion of a third coding sequence for a desired protein.

30. The expression vector of claim 29 wherein the polynucleotide further comprises the third coding sequence.

31. The expression vector of claim 20 further comprising a papovavirus origin of replication.

32. The expression vector of claim 31 wherein the papovavirus is SV40.

33. The expression vector of claim 29 further comprising a second promoter that regulates transcription of the third coding sequence.

34. The expression vector of claim 33 further comprising the third coding sequence.

35. An expression vector comprising a polynucleotide encoding a fusion protein, wherein the polynucleotide comprises:
a first coding sequence for a 107/402-T antigen;
a second coding sequence comprising codons 640-914 of a human progesterone receptor coding sequence, wherein the 5′-most codon of the second coding sequence is located directly 3′ of codon 673 of the first coding sequence;
a promoter that controls transcription of the first and second coding sequences, wherein the promoter is selected from the group consisting of an oncofetal promoter and a tissue-specific promoter;
an internal ribosome entry;
a third coding sequence for a desired protein; and
an SV40 origin of replication.

36. An isolated mammalian cell comprising the expression vector of claim 35.

37. The isolated mammalian cell of claim 36 which is a simian cell.

38. The isolated mammalian cell of claim 36 which is a human cell.

39. An isolated mammalian cell comprising an expression vector, wherein the expression vector comprises a polynucleotide encoding a fusion protein, wherein the polynucleotide comprises:
a first coding sequence for a 107/402-T antigen; and
a second coding sequence for a truncated hormone binding domain of a progesterone receptor, wherein the truncated hormone binding domain binds an antiprogestin but does not bind progesterone, and wherein the 5′-most codon of the second coding sequence is located directly 3′ of codon 673 of the first coding sequence.

40. The isolated mammalian cell of claim 39 which is a simian cell.

41. The isolated mammalian cell of claim 39 which is a human cell.

42. The isolated mammalian cell of claim 39 wherein the second coding sequence comprises codons 640-891 of a human progesterone receptor coding sequence.

43. The isolated mammalian cell of claim 39 wherein the second coding sequence consists of codons 640-891 of a human progesterone receptor coding sequence.

44. The isolated mammalian cell of claim 39 wherein the second coding sequence comprises codons 640-914 of a human progesterone receptor coding sequence.

45. The isolated mammalian cell of claim 39 wherein the second coding sequence consists of codons 640-914 of a human progesterone receptor coding sequence.

46. The isolated mammalian cell of claim 39 wherein the expression vector further comprises a first promoter that regulates transcription of the polynucleotide.

47. The isolated mammalian cell of claim 46 wherein the first promoter is an oncofetal promoter.

48. The isolated mammalian cell of claim 46 wherein the first promoter is a tissue-specific promoter.

49. The isolated mammalian cell of claim 39 wherein the polynucleotide further comprises:
an internal ribosome entry site; and
a restriction enzyme site for insertion of a third coding sequence for a desired protein.

50. The isolated mammalian cell of claim 49 wherein the polynucleotide further comprises the third coding sequence.

51. The isolated mammalian cell of claim 39 wherein the expression vector further comprises a papovavirus origin of replication.

52. The isolated mammalian cell of claim 51 wherein the papovavirus is SV40.

53. The isolated mammalian cell of claim 49 wherein the expression vector further comprises a second promoter that regulates transcription of the third coding sequence.

54. The isolated mammalian cell of claim 53 wherein the expression vector further comprises the third coding sequence.

55. A kit for expressing a desired protein, comprising:
an expression vector comprising a polynucleotide encoding a fusion protein, wherein the polynucleotide comprises:
a first coding sequence for a 107/402-T antigen; and
a second coding sequence for a truncated hormone binding domain of a progesterone receptor, wherein the truncated hormone binding domain binds an antiprogestin but does not bind progesterone, wherein the 5′-most codon of the second coding sequence is located directly 3′ of codon 673 of the first coding sequence.

56. The kit of claim 55 wherein the second coding sequence comprises codons 640-891 of a human progesterone receptor coding sequence.

57. The kit of claim 55 wherein the second coding sequence consists of codons 640-891 of a human progesterone receptor coding sequence.

58. The kit of claim 55 wherein the second coding sequence comprises codons 640-914 of a human progesterone receptor coding sequence.

59. The kit of claim 55 wherein the second coding sequence consists of codons 640-914 of a human progesterone receptor coding sequence.

60. The kit of claim 55 wherein the expression vector further comprises a first promoter that regulates transcription of the polynucleotide.

61. The kit of claim 60 wherein the first promoter is an oncofetal promoter.

62. The kit of claim 60 wherein the first promoter is a tissue-specific promoter.

63. The kit of claim 55 wherein the expression vector further comprises:
an internal ribosome entry site; and
a restriction enzyme site for insertion of a third coding sequence for a desired protein.

64. The kit of claim 63 wherein the expression vector further comprises the third coding sequence.

65. The kit of claim 55 wherein the expression vector further comprises a papovavirus origin of replication.

66. The kit of claim 65 wherein the papovavirus is SV40.

67. The kit of claim 63 wherein the expression construct further comprises a second promoter that regulates transcription of the third coding sequence.

68. The kit of claim 67 wherein the expression construct further comprises the third coding sequence.

69. A kit for expressing a desired protein, comprising:
a human cell comprising an expression vector, wherein the expression vector comprises a polynucleotide encoding a fusion protein, wherein the polynucleotide comprises:
a first coding sequence for a 107/402-T antigen; and
a second coding sequence comprising codons 640-914 of a human progesterone receptor coding sequence, wherein the 5′-most codon of the second coding sequence is located directly 3′ of codon 673 of the first coding sequence;
a promoter that controls transcription of the first and second coding sequences, wherein the promoter is selected from the group consisting of an oncofetal promoter and a tissue-specific promoter;
an internal ribosome entry site; and
a third coding sequence for a desired protein.

70. A kit for expressing a desired protein, comprising:
a mammalian cell comprising an expression vector, wherein the expression vector comprises a polynucleotide encoding a fusion protein, wherein the polynucleotide comprises:
a first coding sequence for a 107/402-T antigen; and
a second coding sequence for a truncated hormone binding domain of a progesterone receptor, wherein the truncated hormone binding domain binds an antiprogestin but does not bind progesterone, wherein the 5′-most coding of the second coding sequence is located directly 3′ of codon 673 of the first coding sequence.

71. The kit of claim 70 wherein the expression vector further comprises a papovavirus origin of replication.

72. The kit of claim 70 wherein the mammalian cell is a simian cell.

73. The kit of claim 70 wherein the mammalian cell is a human cell.

74. A method of expressing a desired protein, comprising the steps of:
culturing a mammalian cell under conditions whereby the desired protein can be expressed, wherein the mammalian cell comprises an expression vector comprising a polynucleotide comprising (1) a first coding sequence for a 107/402-T antigen, (2) a second coding sequence for a truncated hormone binding domain of a progesterone receptor, wherein the truncated hormone binding domain binds an antiprogestin but does not bind progesterone, wherein the 5′-most codon of the second coding sequence is located directly 3′ of codon 673 of the first coding sequence, and (3) a third coding sequence for the desired protein; and
contacting the mammalian cell with an antiprogestin, whereby the desired protein is expressed.

75. The method of claim 74 wherein the mammalian cell is a simian cell.

76. The method of claim 74 wherein the mammalian cell is a human cell.

77. The method of claim 74 further comprising the step of varying the concentration of the antiprogestin over time.

78. The method of claim 74 wherein the antiprogestin is RU486.

79. The method of claim 74 wherein the second coding sequence comprises codons 640-891 of a human progesterone receptor coding sequence.

80. The method of claim 74 wherein the second coding sequence comprises codons 640-914 of a human progesterone receptor coding sequence.

81. The method of claim 74 wherein the expression vector further comprises a papovavirus origin of replication.

82. The method of claim 81 wherein the papovavirus is SV40.

83. A method of expressing a desired protein, comprising the step of:
contacting a mammalian cell with an antiprogestin, wherein the mammalian cell comprises an expression vector comprising a polynucleotide comprising (1) a first coding sequence for a 107/402-T antigen, (2) a second coding sequence for a truncated hormone binding domain of a progesterone receptor, wherein the truncated hormone binding domain binds an antiprogestin but does not bind progesterone, wherein the 5′-most codon of the second coding sequence is located directly 3′ of codon 673 of the first coding sequence, and (3) a third coding sequence for the desired protein, whereby the desired protein is expressed.

84. The method of claim 74 wherein the mammalian cell is a simian cell.

85. The method of claim 74 wherein the mammalian cell is a human cell.

86. The method of claim 74 further comprising the step of varying the concentration of the antiprogestin over time.

87. The method of claim 74 wherein the antiprogestin is RU486.

88. The method of claim 74 wherein the second coding sequence comprises codons 640-891 of a human progesterone receptor coding sequence.

89. The method of claim 74 wherein the second coding sequence comprises codons 640-914 of a human progesterone receptor coding sequence.

90. The method of claim 74 wherein the expression vector further comprises a papovavirus origin of replication.

91. The method of claim 81 wherein the papovavirus is SV40.
